(19) United States
(12) Patent Application Publication
(10) Pub. No.: US 2013/0058863 A1
(43) Pub. Date: Mar. 7, 2013

(54) 4-Protein Biomarker Panel for the Diagnosis of Lymphoma from Biospecimen

(71) Applicant: Jake Yue Chen, Indianapolis, IN (US)

(72) Inventor: Jake Yue Chen, Indianapolis, IN (US)

(21) Appl. No.: 13/576,877

(22) PCT Filed: Feb. 4, 2011
(86) PCT No.: PCT/US11/23742

§ 371 Date: Oct. 24, 2012

Related U.S. Application Data
(60) Provisional application No. 61/301,509, filed on Feb. 4, 2010.
(60) Provisional application No. 61/301,520, filed on Feb. 4, 2010.

(51) Int. Cl. C40B 30/04; C40B 40/10; G06F 17/30; A61P 35/02; A61K 51/10; A61P 35/00; C40B 40/06; A61K 35/14

(52) U.S. Cl. 424/1.49; 506/16; 506/18; 506/9; 424/93.71; 707/705; 707/E17.005

(57) ABSTRACT

A panel of lymphoma related biomarkers are provided. The panel allows the identification of a subject at risk for a lymphoma. Further provided are methods of optimizing therapeutic efficacy associated with treatment of a lymphoma related disorder. Methods of identifying biomarkers affiliated with a condition of interest are provided.

### CROSS-REFERENCE TO RELATED APPLICATIONS

This application claims priority to, and the benefit of U.S. Provisional Patent Application No:61/301,520, filed on Feb. 4, 2010 and U.S. Provisional Patent Application No:61/301,509, filed on Feb. 4, 2010, which are incorporated herein by reference in their entirety.

### FIELD OF THE INVENTION

The present invention relates to the field of evaluating compounds indicative of lymphoma related disorders, classifying lymphoma related disorders and optimizing therapeutic regimens.

### BACKGROUND OF THE INVENTION

Despite the surge in molecular knowledge and the completion of the human genome project, development and identification of biomarkers for clinical use has been a disappointment. Relatively few single molecules highly specific to a condition of interest have been identified. For complex human diseases such as cancer, the etiology of phenotypically similar cancers can rise from completely different molecular mechanisms. This phenomenon may be further complicated by uncertain environmental risks, genetic risks, diet, and lifestyle choices of individuals. Thus identifying single biomarkers or panels of biomarkers specific to a disorder of interest has been considered difficult to achieve.

Recent biomarker studies concerning cancer have suggested that molecular interaction networks can be critical in helping prioritize single biomarkers and multiple biomarker panels. For example, concerning breast cancer, a recent study identified the hyaluronan-mediated motility receptor gene (HMMR) as a new susceptibility locus for breast cancer by first constructing a human protein interaction network for breast cancer susceptibility using several omics data sets; and another study reported that integrating protein-protein interaction network and gene expression information in breast cancer led to several biomarker panels, each containing a small activated subnetwork that can improve prediction of breast cancer metastasis. Both studies suggest that molecular interaction networks, which contain biological functional context information of genes, should become an integral step of multi-biomarker panel development to increase chances of success.

Another study investigated the relationships between human diseases and genetic markers (disease-causing genes) to build a network of disease disorders and disease genes linked by known disorder-gene associations from the Online Mendelian Inheritance in Man (OMIM) database, a database of human genes and genetic disorders. The study indicates that most human diseases are related to each other in a disease association network and many diseases share common genetic origins. The discovery is truly a “double-edged sword” to bioinformaticians interested in biomarker discovery: on the one hand, this suggests that sensitive biomarkers for a new disease of interest may be discovered by borrowing gene or protein biomarkers known to play roles in similar diseases; on the other hand, involvement of genes or proteins in multiple disease processes decreases specificity of candidate biomarkers.

Graph and network visualization is widely accepted in the scientific research community as an essential tool for exploring the complex connections and interactions among data entities and to investigate the inherent structures and knowledge in a broad range of domains. However, several problems have long hampered graph and network visualization. First, the viewing platform and performance pose constraints on the scale of the graphs. Only a few systems can handle large graphs of up to several thousand nodes. Second, visual usability and clarity become unacceptable as the density of the graph grows significantly, even though a system can layout and display this large graph. Nodes and edges occlude each other and are often indiscernible, owing to congestion of color, metaphors, and labels.

In the real world, the data entities and their relationships can be correlated yet heterogeneous. For example, in biology networks, nodes could be cDNA, enzymes, chemicals, organs and diseases, and the relationships among data entities could represent a variety of biological processes. To model these data entities in a single large graph, there is a great demand to encode different aspects of information, onto the limited space on and around nodes and links. Inappropriate modeling does not only aggravate the congestions in large scale networks, but is also likely to miss the knowledge inherently due to the correlations among different categories.

Information visualization techniques have played central roles in exposing change patterns of thousands of parallel molecular measurements in genomic, functional genomics, and proteornics data derived from disease samples. Graph and network visualization tools are becoming essential for biologists and biochemists who study bio-molecular interaction networks, including protein interaction networks, gene regulatory networks, and metabolic networks. Several biomolecular interaction databases, for example DIP, BIND and Reactome, have become available, fueling the growing need for the study of the functional relationships among genes/proteins in network contexts. While using the graph metaphor for visualizing biomolecular networks is appropriate for understanding the basic topological structure of biomolecular networks, or in some cases, high-level protein categorical interconnections in a network, the metaphor is inadequate in addressing biological determinations in which correlated functional changes of genes, proteins, and metabolites have to be investigated in the same network context. Examples of these determinations include, for example, determining the significant gene expression pattern changes in a given biological condition such as human disease; determining the functional relevance of such changes; and ‘seeing’ biologically significant changes in gene/protein expression measurements, despite inherent data noise from DNA microarray experiments. These determinations can be of central concern in post-genome molecular diagnostics applications, particularly molecular biomarker discoveries. Conventional graph-based network visualization methods are often insufficient in addressing these post-genome biological knowledge discovery determinations. It would be desirable to have an information visualization technique that can capture, display and process large amounts of information and present it in a way that enables researchers to understand the processes represented by the data.

Lymphomas are diagnosed in more than 50,000 new patients in the United States each year. Presentation of a lymphoma may resemble presentation of a leukemia. Thus, it is difficult to differentiate lymphomas such as Hodgkin's disease from lymphadenopathy caused by other disorders such as leukemia (see Beers & Berkow, Eds., Merck Manual of Diagnosis and Therapy, 17^{th} Edition, 1999, Merck Research Laboratories, Whitehouse Station N.J., ch. 139).

### SUMMARY OF THE INVENTION

Compositions and methods useful for classifying lymphoma related disorders are provided. The inventions are based on the surprising discovery that evaluating expression of a lymphoma related biomarker panel comprising four biomarkers, TNFRSF8, FSCN1, BCL6 and PIM1, is significantly more informative than evaluating expression of the individual biomarkers, TNFRSF8, FSCN1, BCL6 and PIM1. Altered expression of the lymphoma related biomarker panel indicates lymphoma and allows distinction between a lymphoma and a leukemia. Accurate classification of a subject at risk for a lymphoma related disorder as being at risk for a lymphoma or at risk for a leukemia allows optimization of therapeutic regimens and reduces exposure of a subject to the side effects from administration of a less effective treatment regimen.

Compositions provided herein include kits for evaluating expression of at least three biomarkers from a lymphoma related biomarker panel that comprises TNFRSF8, FSCN1, BCL6 and PIM1. A kit provided herein comprises a first biomarker detection reagent capable of preferentially detecting expression of a first biomarker selected from the lymphoma related biomarker panel, a second biomarker detection reagent capable of preferentially detecting expression of a second biomarker selected from the lymphoma related biomarker panel, and a third biomarker detection reagent capable of preferentially detecting expression of a third biomarker selected from the lymphoma related biomarker panel. In an aspect of the kit, the kit further comprises a fourth biomarker detection reagent capable of preferentially detecting expression of a fourth biomarker selected from the lymphoma related biomarker panel. In another aspect of the kit, the first biomarker detection reagent preferentially detects expression of TNFRSF8, the second biomarker detection reagent preferentially detects expression of FSCN1, the third biomarker detection reagent preferentially detects expression of BCL6 and the fourth biomarker detection reagent preferentially detects expression of PIM1.

Kits for characterizing a lymphoma related disorder in a subject at risk for a lymphoma related disorder comprising at least three biomarker detection reagents for at least three biomarkers from a lymphoma related biomarker panel that comprises TNFRSF8, FSCN1, BCL6 and PIM1 are provided. Such a kit provided herein comprises a first biomarker detection reagent capable of preferentially detecting expression of a first biomarker selected from the lymphoma related biomarker panel, a second biomarker detection reagent capable of preferentially detecting expression of a second biomarker selected from the lymphoma related biomarker panel, and a third biomarker detection reagent capable of preferentially detecting expression of a third biomarker selected from the lymphoma related biomarker panel. In an aspect of the kit, the kit further comprises a fourth biomarker detection reagent capable of preferentially detecting expression of a fourth biomarker selected from the lymphoma related biomarker panel. In another aspect of the kit, the first biomarker detection reagent preferentially detects expression of TNFRSF8, the second biomarker detection reagent preferentially detects expression of FSCN1, the third biomarker detection reagent preferentially detects expression of BCL6 and the fourth biomarker detection reagent preferentially detects expression of PIM1.

Methods of characterizing a lymphoma related disorder in a subject at risk for a lymphoma related disorder comprising the steps of providing a biological sample obtained from the subject; evaluating expression in the sample of at least three biomarkers from a lymphoma related biomarker panel comprising TNFRSF8, FSCN1, BCL6 and PIM1; comparing the expression of the biomarkers with a predetermined standard; identifying the biomarker expression as altered or unaltered and characterizing the lymphoma related disorder as lymphoma when the expression of the biomarkers is altered. In an aspect of the methods, the methods comprise evaluating expression in the sample of at least four biomarkers from a lymphoma related biomarker panel. In another aspect of the methods, at least three of the biomarkers are selected from the group consisting of TNFRSF8, FSCN1, BCL6 and PIM1. In various aspects of the methods, the subject is a mammal or a mammal selected from the group comprising humans, bovines, equines, murines, ovines, caprines, lapines, canines and swine. Another aspect of the methods provides that the Type I error rate is less than 20%. Yet another aspect of the methods provides that the Type II error rate is less than 20%. In aspects of the methods, the altered expression of each biomarker differs from the predetermined standard by at least 0.001%. The altered expression may be decreased expression or increased expression.

Methods of optimizing therapeutic efficacy associated with treatment of a lymphoma related disorder in a subject at risk for a lymphoma related disorder are provided. Such methods comprise the steps of providing a biological sample obtained from the subject, evaluating expression in the sample of at least three biomarkers from a lymphoma related biomarker panel comprising TNFRSF8, FSCN1, BCL6 and PIM1, comparing expression of the biomarkers with a predetermined standard, identifying expression of the biomarkers as altered or unaltered, and administering a lymphoma preferred course of treatment to the subject when expression of the biomarkers in the panel is altered. Aspects of the methods include evaluating expression in the sample of at least four biomarkers in the lymphoma related biomarker panel. In various aspects of the methods at least three of the biomarkers are selected from the group consisting of TNFRSF8, FSCN1, BCL6 and PIM1.

Methods of optimizing therapeutic efficacy associated with treatment of a lymphoma related disorder in a subject at risk for a lymphoma related disorder are provided. Such methods comprise the steps of providing a biological sample obtained from the subject, evaluating expression in the sample of at least three biomarkers from a lymphoma related biomarker panel comprising TNFRSF8, FSCN1, BCL6 and PIM1, comparing expression of the biomarkers with a predetermined standard, identifying expression of the biomarkers as altered or unaltered, and administering a leukemia preferred course of treatment to the subject when expression of the biomarkers in the panel is unaltered. Aspects of the methods include evaluating expression in the sample of at least four biomarkers in the lymphoma related biomarker panel. In various aspects of the methods at least three of the biomarkers are selected from the group consisting of TNFRSF8, FSCN1, BCL6 and PIM1.

A visualization method for determination of candidate biomarker panels for a disease of interest is disclosed. The visualization method includes accessing a protein database containing data regarding genes and protein, and accessing a disease database containing data regarding diseases. The visualization method also includes constructing a protein base network and protein terrain using the data from the protein database for a disease of interest, and displaying the protein terrain on a computer display device. The visualization method also includes constructing a disease base network and disease terrain using the data from the disease database for the proteins of the protein base network, and displaying the disease terrain on a computer display device. The constructing of the base networks and terrains is done with a computer processor. The method then includes determining a candidate biomarker panel using the displayed protein terrain and the displayed disease terrain.

### BRIEF DESCRIPTION OF THE DRAWINGS

FIG. 1 illustrates an exemplary overview of terrain visualization panel construction for a Molecular Network Terrain and a Phenotypic Network Terrain.

FIG. 2 illustrates an exemplary iterative refinement process for biomarker development using terrain visualization panels.

FIG. 3 shows a three-dimensional terrain derived from a two-dimensional base network, the corresponding base network, and an exemplary contour map.

FIG. 4 shows exemplary pseudocode for laying out a base network.

FIG. 5(a) shows a schematic arrangement of a terrain surface on top of a node in a cancer term network.

FIG. 5(b) shows the formation of the terrain surface in FIG. 5(a) with a gene term network as the base network.

FIG. 6A shows gene terrains arranged on a core gene network.

FIG. 6B includes a Panel B with detailed views of four of the gene terrains shown in FIG. 6A, a Panel C showing three disease terrains formed into a cluster; a Panel D showing terrains of major cancer terms identified by observing gene terrains shown in FIG. 6A, and a heatmap with rows for cancers and columns for genes.

FIG. 7 shows molecular network terrains for each of breast cancer, ovarian cancer, and lung cancer, respectively, varied among four types of protein interaction base networks of increasing quality, HAPPI-2, HAPPI-3, HAPPI-4 and HAPPI-5.

FIG. 8 shows disease terrains developed for four cancer biomarkers well-documented in the literature to examine their disease biomarker specificity as potential candidate biomarkers for detection of prostrate cancer and ovarian cancer.

FIG. 9 shows the protein identifier, rank and calculated Alzheimer's disease relevance gene ranking score for the top twenty significant proteins to Alzheimer's disease.

FIG. 10A shows the Alzheimer's disease gene terrain base network layout before optimization.

FIG. 10B shows the Alzheimer's disease gene terrain base network layout after optimization.

FIG. 11A shows an exemplary terrain surface indicating gene expression data from the Alzheimer's disease normal (control) group.

FIG. 11B shows an exemplary contour indicating gene expression data from the Alzheimer's disease normal (control) group.

FIG. 12A shows the exemplary terrain surface of FIG. 11A with a protein threshed by T=3.

FIG. 12B shows a contour visualization of the exemplary terrain surface of FIG. 12A.

FIG. 13A shows a zoomed-in view of a portion of the contour of FIG. 12B.

FIG. 13B shows a further zoomed-in view of a portion of the contour of FIG. 12B.

FIG. 14A shows a differential expression terrain surface for control versus incipient condition for Alzheimer's disease.

FIG. 14B shows a differential expression contour for control versus incipient condition for Alzheimer's disease.

FIG. 15A shows a differential expression terrain surface for control versus moderate condition for Alzheimer's disease.

FIG. 15B shows a differential expression contour for control versus moderate condition for Alzheimer's disease.

FIG. 16A shows a differential expression terrain surface for control versus severe condition for Alzheimer's disease.

FIG. 16B shows a differential expression contour for control versus severe condition for Alzheimer's disease.

FIG. 17A shows the results of interactive visual querying, in which the name of proteins in the peak or valleys with differential gene expression levels above thresholds in control versus incipient Alzheimer's disease is shown.

FIG. 17B displays a contour map corresponding to FIG. 17A.

FIG. 18 shows a four-step approach to iteratively design panel biomarkers that includes a construction step, a filtering step, an evaluation step, and a rendering step.

FIG. 19 shows a sequence of protein terrains and contour visualizations in a correlative visual analysis for a lymphoma case study using the approach outlined in FIG. 18; The initial pool of candidate cancer biomarkers was 762. The first filtering step yielded 169 candidate lymphoma related biomarkers from the starting pool of 762. The second filtering step refined the 169 candidate lymphoma related biomarkers to 31 candidate lymphoma related biomarkers. Finally iterative refinement of the 31 candidate lymphoma related biomarkers yielded lymphoma related biomarker panel comprising four biomarkers: TNFRSF8, FSCN1, BCL6 and PIM1.

FIG. 20 shows an exemplary operating environment comprising several computer systems that are coupled together through a network.

FIG. 21 shows an exemplary computer system that can be used as a client computer system or a server computer system or as a web server system.

FIG. 22 presents two panels assessing a lymphoma related biomarker panel comprising TNFRSF8, FSCN1, BCL6 and PIM1. Panel A presents cumulative distribution plots (CDF) of Type I (dashed line) and Type II (dotted line) error rates of the lymphoma related panel and the pool of 152 other lymphoma related molecules (benchmark molecules). Y value presents the portion of the benchmark population whose error rates are equal to or less than x. Crosses in the cumulative distribution line and vertical lines indicate the error rate of the individual biomarkers from the lymphoma related biomarker panel: TNFRSF8 is indicated with “A”, FSCN1 is indicated with “B”, BCL6 is indicated with “C”, and PIM1 is indicated with “D”. The Type I error rate of the biomarker panel (circle on the cumulative distribution line) is 0.0069, significantly less than 1%. The Type I error rates that occurs for each of TNFRSF8, FSCN1 and BCL6 are significantly higher than the Type I error rate that occurs when evaluating all four members of the lymphoma related biomarker panel. The panel's type I error rate is larger than that of Pim1 alone. An enlarged view of the Type II error profile is presented in the inset panel. The lymphoma related biomarker panel comprising TNFRSF8, FSCN1, BCL6 and PIM1 has a Type II error rate much less than 1% level. The y-axis value indicates that the Type II error rate of the panel has a relative top 9% ranking in the benchmark pool of 152 other lymphoma related molecules. The panel's Type II error rate is lower than that of each of PIM1, TNFRSF8, and BCL6 individually. The panel's Type II error rate is higher than that of FSCN1 individually. The combined results of the Type I and Type II error rates of the lymphoma related biomarker panel outperforms each of the four underlying component molecules.

FIG. 22, Panel B presents cumulative distribution plots (CDF) of disease specificity. The x value is the relative ranking in the benchmark population, and Y value is the percentage of lymphoma samples in lymphoma-dominated classes. TNFRSF8 is indicated with “A”, FSCN1 is indicated with “B”, BCL6 is indicated with “C”, and PIM1 is indicated with “D”. The y value of the biomarker panel (cross, vertical line labeled X) is 0.9914 or larger than 99%. The four biomarker panel comprising TNFRSF8, FSCN1, BCL6 and PIM1 outperforms any component molecule.

### DETAILED DESCRIPTION OF THE INVENTION

The application provides kits for evaluating expression of at least three biomarkers from a lymphoma related biomarker panel comprising TNFRSF8, FSCN1, BCL6 and PIM1. Kits for characterizing a lymphoma related disorder in a subject at risk for a lymphoma related disorder comprising at least three biomarkers from a lymphoma related biomarker panel comprising TNFRSF8, FSCN1, BCL6 and PIM1 are also provided. Further provided are methods of characterizing a lymphoma related disorder in a subject at risk for a lymphoma related disorder, methods of optimizing therapeutic efficacy associated with treatment of a lymphoma related disorder, and methods of identifying a subject at risk for a lymphoma related disorder. Kits and methods of the present application may be used to validate new lymphoma-related biomarkers or new lymphoma related assays. The compositions and methods were developed from investigations that revealed that a lymphoma related biomarker panel comprising TNFRSF8, FSCN1, BCL6 and PIM1 exhibits an improved total error rate and high specificity for lymphoma rather than leukemia.

The phrase “lymphoma related disorder” is intended to encompass a lymphoma, leukemia, or a symptomatically similar disorder. Symptoms of a lymphoma or leukemia include, but are not limited to, anemia, thrombocytopenia, granulocytopenia, hepatomegaly, splenomegaly, enlarged lymph nodes, enlargement of kidneys or gonads, cranial nerve palsies, abnormal red blood cell (RBC) morphology, abnormal cytochemical appearance, bone marrow failure, granulocytic sarcomas, chloromas, altered immunophenotype, abnormal white blood cell (WBC) concentration, differential white blood cell concentration, altered platelet concentration, lymphadenopathy, splenomegaly, hemolytic anemia, Auer rod presence, hypogammaglobulinemia, hemolytic anemia, fatigue, fever, malaise, weight loss, petechiae, epistaxis, menstrual irregularity, easy bruisability, bone pain, joint pain; abnormal staining with terminal transferase, myeloperoxidase, Sudan black B, specific esterase, and non-specific esterase; abnormal histochemical stains; excessive bleeding; abnormal karyotypes, B-cell immunophenotype, testis swelling, disseminated intravascular coagulation (DIC), neutropenia, decreased immunoglobulin production, fatigue, anorexia, weight loss, dyspnea on exertion, pallor, lymphocytocis, increased lymphocytes in the bone marrow, excessive granulocyte production, myelofibrosis, night sweats, abnormal leukocyte alkaline phosphatase score, siderofibroblast presence, altered basophil concentrations, leukocytosis, basophilia, eosinophilia, abnormal cell morphology, hematopoietic cell proliferation, macrocytosis, anisocytosis, altered platelet morphology, pseudo-Pelger Huët cell presence, abnormal neutrophil cytoplasmic granularity, hypercellular bone marrow, Reed-Sternberg cell presence, heterogeneous background cellular infiltrate, cervical adenopathy, mediastinal adenopathy, pruritis, Pel-Ebstein fever, pain post alcohol consumption, vertebral osteoblastic lesions, back pain, osteolytic lesions, compression fractures, panctyopenia, paraplegia, Horner's syndrome, laryngeal paralysis, neuralgia, jaundice, edema, wheezing, lobar consolidation, bronchopneumonia, cavitation, lung abscess, impaired immune response, cachexia, thrombocytosis, abnormal serum alkaline phosphatase levels, CD15 and TNFRSF8 cell status, skin infiltrates, malignant T cells, hypercalcemia; rubbery, discrete or matted lymph nodes; chylous ascites, pleural effusion, congestion, renal failure, lymph node architecture modification, CD45 presence, elevated mitotic rate, altered pathology, and starry sky pattern.

The term “lymphoma” is intended to encompass a heterogeneous group of neoplasms arising in either the reticuloendothelial or lymphatic systems. Lymphomas include, but are not limited to, lymphoblastoid lymphoma, Hodgkin's disease, non-Hodgkin's disease, non-Hodgkin's lymphoma (NHL), mucosa-associated lymphoid tumors (MALT), mantle cell lymphoma, diffuse small cleaved cell lymphoma, anaplastic large cell lymphoma, Ki-1 lymphoma, adult T-cell leukemia-lymphoma, immunoblastic NHL, small noncleaved NHL, Burkitt's lymphoma, K-1 anaplastic large cell lymphoma, diffuse large cell NHL, lymphoblastic NHL, T-cell lymphoblastic lymphoma, mycosis fungoides, and Sezary syndrome.

The word “leukemia” is intended to encompass a malignant neoplasm of a blood-forming tissue or tissues. Leukemias include but are not limited to, acute leukemias such as but not limited to, acute lymphoblastic leukemia (ALL), acute lymphocytic leukemia, acute myelogenous leukemia (AML), acute myeloid leukemia, acute myelocytic leukemia, acute promyelocytic leukemia (APL), chronic leukemias such as but not limited to, chronic lymphocytic leukemia (CLL), chronic lymphatic leukemia, B-cell CLL, T-cell CLL, prolymphocytic leukemia, hairy cell leukemia, chronic myelocytic leukemia, chronic myeloid leukemia, chronic myelogenous leukemia, chronic myelomonocytic and chronic granulocytic leukemia.

Kits and methods of the application may involve evaluating expression of at least a first biomarker, second biomarker and third biomarker selected from a lymphoma related biomarker panel comprising TNFRSF8, FSCN1, BCL6 and PIM1 and may involve evaluating expression of a fourth biomarker selected from a lymphoma related biomarker panel comprising TNFRSF8, FSCN1, BCL6 and PIM1. Kits and methods of the application involve evaluating expression of at least a first biomarker, second biomarker and third biomarker selected from the group consisting of TNFRSF8, FSCN1, BCL6 and PIM1 and may involve evaluating expression of a fourth biomarker selected from the group consisting of TNFRSF8, FSCN1, BCL6 and PIM1. Kits and methods of the application may involve evaluating expression of additional biomarkers selected from a lymphoma related biomarker panel.

The phrase “biomarker” encompasses a distinctive biological or biologically derived indicator of a process, event or condition. A biomarker may be a biological compound such as but not limited to, a protein, polypeptide, peptide, nucleic acid molecule, metabolite, compound, antigen, antigenic fragment, glycoprotein, lipoprotein, enzyme, hormone, carbohydrate and fragments thereof of which the presence, absence, concentration, or location in a subject yields information relevant to a particular condition, process or event. In various embodiments the application provides compositions and methods for evaluating expression of a biomarker. It is recognized that any means of evaluating expression known in the art may be utilized in the methods; it is also recognized that methods of evaluating expression at the mRNA level may differ from methods of evaluating expression at the polypeptide or peptide level. Methods of evaluating expression are described elsewhere herein.

A “panel”, “group”, or “library” of related biomarkers comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55-60, 60-65, 65-70, 70-75, 75-80, 80-85, 85-90, 90-95, 95-100, or 100 or more related biomarkers. The phrase “lymphoma related biomarker panel” is intended to encompass a biomarker panel comprising biomarkers linked to lymphoma, leukemia or a symptomatically similar disorder. It is envisioned that each lymphoma related biomarker in a panel may be assayed by a distinct method or by similar methods. In non-limiting examples each compound in panel may be assayed by the same method, one compound may be assayed by one method while the remainder are assayed by a different method, two or more compounds in the panel may be assayed by one method while the remainder are assayed by a different method, two or more compounds in the panel may be assayed by distinct methods while the remainder are assayed by one similar method, or each compound may be assayed by a distinct method. A preferred lymphoma related biomarker panel of the instant application comprises at least three lymphoma related biomarkers selected from the group consisting of TNFRSF8, FSCN1, BCL6 and PIM1. Another preferred lymphoma related biomarker panel of the instant application comprises at least four lymphoma related biomarkers selected from the group consisting of TNFRSF8, FSCN1, BCL6 and PIM1.

“TNFRSF8”, also known as TNR8, TNFR8, Tumor Necrosis Factor Receptor Superfamily 8, CD30, CD30L receptor, Ki-1 antigen, lymphocyte activation antigen CD-30, CD_antigen=CD30, TNFRSF8, and D1S166E, Uniprot ProtID P28908 and RefSeq ID NM_{—}001234, is intended to encompass a nucleic acid molecule having the nucleotide sequence set forth in SEQ ID NO:1, a nucleic acid molecule having a nucleotide sequence complementary to the nucleotide sequence set forth in SEQ ID NO:1, a polypeptide having the amino acid sequence set forth in SEQ ID NO:2, and a nucleic acid molecule that encodes a polypeptide having the amino acid sequence set forth in SEQ ID NO:2. A TNFRSF8 nucleic acid molecule is a 3686 nucleotide nucleic acid molecule having the nucleotide sequence set forth in SEQ ID NO:1. Preferred fragments of a TNFRSF8 nucleic acid molecule may include but are not limited to, regions of nucleic acid molecules suitable for amplification, suitable primer binding regions and suitable probe binding regions. Fragments of a TNFRSF8 nucleic acid molecule that may be useful in the current methods include fragments comprising up to 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600 or up to 3686 consecutive nucleotides of the sequence set forth in SEQ ID NO:1. A TNFRSF8 polypeptide is a polypeptide having the 595 amino acid sequence set forth in SEQ ID NO:2. Fragments of a TNFRSF8 polypeptide that may be useful in the current methods include fragments comprising up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or up to 595 consecutive amino acids of the sequence set forth in SEQ ID NO:2. Preferred fragments of polypeptides may include but are not limited to antigenic regions, matured fragments, membrane domains, cytosolic domains and fragments that are removed during protein processing.

“FSCN1”, also known as p55, fascin, 55 kDa actin-bundling protein, FAN1, HSN, SNL, singed-like protein, Uniprot ProtID Q16658 and RefSeq ID NM_{—}003088, is intended to encompass a nucleic acid molecule having the nucleotide sequence set forth in SEQ ID NO:3, a nucleic acid molecule having a nucleotide sequence complementary to the nucleotide sequence set forth in SEQ ID NO:3, a polypeptide having the amino acid sequence set forth in SEQ ID NO:4, and a nucleic acid molecule that encodes a polypeptide having the amino acid sequence set forth in SEQ ID NO:4. A FSCN1 nucleic acid molecule is a 2780 nucleotide nucleic acid molecule having the nucleotide sequence set forth in SEQ ID NO:3. Preferred fragments of a FSCN1 nucleic acid molecule may include but are not limited to, regions of nucleic acid molecules suitable for amplification, suitable primer binding regions and suitable probe binding regions. Fragments of a FSCN1 nucleic acid molecule that may be useful in the current methods include fragments comprising up to 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, or up to 2780 consecutive nucleotides of the sequence set forth in SEQ ID NO:3. A FSCN1 polypeptide is a polypeptide having the 493 amino acid sequence set forth in SEQ ID NO:4. Fragments of a FSCN1 polypeptide that may be useful in the current methods include fragments comprising up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or up to 493 consecutive amino acids of the sequence set forth in SEQ ID NO:4. Preferred fragments of polypeptides may include but are not limited to antigenic regions, matured fragments, phosphorylation regions and fragments that are removed during protein processing.

“BCL6”, also known as B-cell lymphoma 6 protein, BCL-6, protein LAZ-3, B-cell lymphoma 5 protein, BCL-5, Zinc-finger and BTB domain containing protein 27, Zinc finger protein 51, ZBTB27, ZNF51, Uniprot ProtID P41182 and RefSeq ID NM_{—}001706, is intended to encompass a nucleic acid molecule having the nucleotide sequence set forth in SEQ ID NO:5, a nucleic acid molecule having a nucleotide sequence complementary to the nucleotide sequence set forth in SEQ ID NO:5, a polypeptide having the amino acid sequence set forth in SEQ ID NO:6, and a nucleic acid molecule that encodes a polypeptide having the amino acid sequence set forth in SEQ ID NO:6. A BCL6 nucleic acid molecule is a 3579 nucleotide nucleic acid molecule having the nucleotide sequence set forth in SEQ ID NO:5. Preferred fragments of a BCL6 nucleic acid molecule may include but are not limited to, regions of nucleic acid molecules suitable for amplification, suitable primer binding regions and suitable probe binding regions. Fragments of a BCL6 nucleic acid molecule that may be useful in the current methods include fragments comprising up to 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, or up to 3579 consecutive nucleotides of the sequence set forth in SEQ ID NO:5. A BCL6 polypeptide is a polypeptide having the 706 amino acid sequence set forth in SEQ ID NO:6. Fragments of a BCL6 polypeptide that may be useful in the current methods include fragments comprising up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, or up to 706 consecutive amino acids of the sequence set forth in SEQ ID NO:6. Preferred fragments of polypeptides may include but are not limited to antigenic regions, matured fragments, dimerization domains, phosphorylation regions, DNA binding domains and fragments that are removed during protein processing.

“PIM1”, also known as, proto-oncogene serine/threonine protein kinase pim-1, pim-1 oncogene, Uniprot ID P11309 and RefSeq ID NM_{—}002648, is intended to encompass a nucleic acid molecule having the nucleotide sequence set forth in SEQ ID NO:7, a nucleic acid molecule having a nucleotide sequence complementary to the nucleotide sequence set forth in SEQ ID NO:7, a polypeptide having the amino acid sequence set forth in SEQ ID NO:8, and a nucleic acid molecule that encodes a polypeptide having the amino acid sequence set forth in SEQ ID NO:8. A PIM1 nucleic acid molecule is a 2751 nucleotide nucleic acid molecule having the nucleotide sequence set forth in SEQ ID NO:7. Preferred fragments of a PIM1 nucleic acid molecule may include but are not limited to, regions of nucleic acid molecules suitable for amplification, suitable primer binding regions and suitable probe binding regions. Fragments of a PIM1 nucleic acid molecule that may be useful in the current methods include fragments comprising up to 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, or up to 2708 consecutive nucleotides of the sequence set forth in SEQ ID NO:7. A PIM1 polypeptide is a polypeptide having the 404 amino acid sequence set forth in SEQ ID NO:8. Fragments of a PIM1 polypeptide that may be useful in the current methods include fragments comprising up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or up to 404 consecutive amino acids of the sequence set forth in SEQ ID NO:8. Preferred fragments of polypeptides may include but are not limited to antigenic regions, matured fragments, ATP binding sites, phosphorylation regions, and fragments that are removed during protein processing.

Kits for evaluation expression of biomarkers from a lymphoma related biomarker panel and for characterizing a lymphoma related disorder are provided herein. A kit of the present application comprises at least three biomarker detection reagents for at least three biomarkers from a lymphoma related biomarker panel and selected from the group consisting of TNFRSF8, FSCN1, BCL6 and PIM1. It is recognized that a kit of the instant application may provide biomarker detection reagents suitable for use in any method of preferentially evaluating expression of a biomarker of interest. It is further recognized that a kit may provide biomarker detection reagents suitable for use in different methods of evaluating expression. In a preferred embodiment, the biomarker detection reagents for the biomarkers of interest may be used in the same method of evaluating expression. It is recognized that the claimed kits and methods may involve multiple methods of evaluating expression of the biomarkers of interest.

A “detection reagent” is an agent or compound that preferentially interacts with or preferentially detects a biomarker of interest. Such detection reagents may include, but are not limited to, an antibody, polyclonal antibody, or monoclonal antibody that preferentially binds a biomarker of interest; an isolated nucleic acid molecule that complements a biomarker of interest such as a primer pair or probe that preferentially hybridizes to a biomarker of interest, a mass spectrometry (MS) probe, and a substrate to which multiple detection reagents that preferentially interact with one or more biomarkers of interest are attached, affixed or connected. Preferred detection reagents are suitable for use in a method of evaluating expression. Kits of the application comprise a detection reagent for a first biomarker, a second biomarker, a third biomarker, and may further comprise a detection reagent for a fourth biomarker; such kits may further comprise a detection reagent for a biomarker including but not limited to a fifth biomarker, a sixth biomarker, a seventh biomarker, an eighth biomarker, a ninth biomarker, a tenth biomarker, a twentieth biomarker or more.

Kits provided herein may comprise a carrier, package or container that is compartmentalized to receive one or more container such as vials, tubes, and the like. A kit provided herein may comprise additional containers comprising materials desirable from a commercial, clinical or user standpoint, including but not limited to, buffers, diluents, filters, needles, syringes, and package inserts with instructions for use. A kit may provide positive or negative controls and may provide a known sample to be used as a predetermined standard. A kit may provide information pertaining to a predetermined standard such as information pertaining to a predetermined range.

A subject “at risk for” a lymphoma related disorder is intended to encompass a subject that has exhibited or is currently exhibiting one or more symptoms of a lymphoma or leukemia, a subject that has a lymphoma or leukemia, a subject that is related to a subject that has exhibited or is currently exhibiting one or more symptoms of a lymphoma or leukemia, a subject that is related to a subject that has a lymphoma or leukemia, a subject that has been exposed an environmental factor related to lymphoma or leukemia development, a subject that has been exposed to a lymphoma or leukemia related virus, and a subject that has received a compound or chemical agent related to lymphoma or leukemia development.

A “biological sample” is intended to encompass a sample collected from a subject including, but not limited to, blood, serum, plasma, tissues, bone marrow, cells, mucosa, fluid, scrapings, hairs, cell lysates, secretions, and urine. Biological samples such as blood and serum samples can be obtained by any method known to one skilled in the art. Suitable subjects include mammals including, but not limited to, primates, humans, equines, bovines, ovines, caprines, porcines, murines, canines, lapines, swine, simians, camelids, domesticated mammals and research mammals.

By “assaying” is intended measuring, quantifying, scoring, or detecting the amount, concentration, or relative abundance of a substance. Methods of evaluating biological compounds are known in the art. It is recognized that a method of assaying one type of biological compound, such as a protein, may not be suitable for assaying another type of biological compound, such as a nucleic acid. It is recognized that methods of assaying a biological compound include direct measurements and indirect measurements. One skilled in the art would be able to select an appropriate method of assaying a particular biological compound.

Methods of assaying biological compounds include, but are not limited to, immunogenic methods, spectrophotometric methods, mass spectroscopy (MS), spectroscopy, GC-MS, MS-MS, X-ray crystallography, NMR, coimmunoprecipitation, FRET, size exclusion chromatography, Western blots, affinity chromatography, thin layer chromatography, HPLC, FPLC, gel filtration chromatography, tandem mass spectrometry, RT-PCR, qualitative Western blot analysis, immunoprecipitation, radiological assays, polypeptide purification, spectrophotometric analysis, Coomassie staining of acrylamide gels, ELISAs, 2-D gel electrophoresis, microarray analysis, in situ hybridization, chemiluminescence, silver staining, enzymatic assays, ponceau S staining, multiplex RT-PCR, immunohistochemical assays, radioimmunoassay, colorimetric analysis, immunoradiometric assays, positron emission tomography, Northern blotting, fluorometric assays, SAGE, ion-intensity based label free quantitative proteomics (LFQP), surface enhanced laser desorption/ionization (SELDI), SELDI-MS, SELDI-TOF, SELDI-TOF-MS, slot blot assay, multi-polar resonance spectroscopy, gas phase ion spectrometry, atomic force microscopy, mass-spectrometry (MS), CD, immunoassays, peptide sequencing, SDS-polyacrylamide gel electrophoresis (SDS-PAGE), electron spray mass spectroscopy, NMR, sedimentation equilibrium, flow cytometry, tandem mass spectrometry, FRET, liquid crystal-MS (LC-MS), MALDI, MALDI-TOV, MALDI-MS, microassays, ion-exchange, reverse phase HPLC, peptide mass fingerprinting (PMF), 2-D DIGE, and microscale solution isoelectrofocusing (MicroSol IEF). See for example McMaster 2005, LCMS a Practical User's Guide, Wiley Interscience; McMaster, 2008, GCMS a Practical User's Guide, Wiley Interscience; Ham, 2008 Even Electron Mass Spectrometry with Biomolecule Applications, Wiley Interscience, Eidhammer et al (2008) Computational Methods for Mass Spectrometry Proteomics, Wiley Interscience; Yan & Chen, 2005, Brief Funct Genomic Proteomics 4:27-38; Zhang et al 2006 J. Proteome Res 5:2909-2918; Wang et al 2006 J. Proteome Res; Ono et al 2006 Mol Cell Proteomics 5:1338-1347; Ausubel et al, eds. (2002) Current Protocols in Molecular Biology, Wiley-Interscience, New York, N.Y.; Coligan et al (2002) Current Protocols in Protein Science, Wiley-Interscience, New York, N.Y.; and Sun et al. (2001) Gene Ther. 8:1572-1579.

A predetermined standard provides a comparison population, comparison group, comparison sample, or a predetermined standard range obtained from a comparison population, comparison group or comparison sample. A predetermined standard range for a biomarker provides a standard range of concentrations, quantities, clinical values, or lab values for the biomarker that is selected, identified, established, or indicated in advance of assaying the level of a biomarker. It is envisioned that predetermined standard ranges for a particular biomarker may vary for different biological samples, that predetermined standard ranges for a particular biomarker may overlap in different biological samples, and that predetermined standard ranges for a particular biomarker may be similar in different biological samples. For example the values of a predetermined standard range for compound x in serum may differ from the values of a predetermined standard range for compound x in urine. It is well within the ability of one skilled in the art to utilize a predetermined standard range suitable for the biological sample being analyzed. It is envisioned that a predetermined standard range encompasses a range between two values, a range equal to or less than a particular value, and a range equal to or greater than a particular value. In an embodiment a predetermined standard range is developed from the levels found in a population of similar subjects, such as healthy, normal or control subjects or subjects with leukemia.

Expression of an individual biomarker that is not within the range of the predetermined standard is identified as altered. Altered expression is an expression level that differs from the predetermined standard range; such a difference, alteration, change or variation encompasses decreased expression and increased expression. It is further recognized that expression of one biomarker may be altered while expression of another biomarker may be unaltered.

Expression is intended to encompass production of any product by a gene including but not limited to transcription of mRNA and translation of polypeptides, peptides, and peptide fragments. “Evaluating expression” encompasses assaying, measuring, quantifying, scoring, or detecting the amount, concentration, or relative abundance of a gene product. It is recognized that a method of evaluating expression of one type of gene product, such as a polypeptide, may not be suitable for assaying another type of gene product, such as a nucleic acid. It is recognized that methods of assaying a gene product include direct measurements and indirect measurements. One skilled in the art would be able to select an appropriate method of evaluating expression of a particular gene product.

Methods of evaluating expression known in the art include, but are not limited to immunogenic methods, spectrophotometric methods, mass spectroscopy (MS), spectroscopy, GC-MS, MS-MS, NMR, FRET, size exclusion chromatography, coimmunoprecipitation, Western blots, affinity chromatography, thin layer chromatography, HPLC, FPLC, gel filtration chromatography, tandem mass spectrometry, RT-PCR, qualitative Western blot analysis, immunoprecipitation, radiological assays, polypeptide purification, spectrophotometric analysis, Coomassie staining of acrylamide gels, ELISAs, 2-D gel electrophoresis, microarray analysis, in situ hybridization, chemiluminescence, silver staining, enzymatic assays, ponceau S staining, multiplex RT-PCR, immunohistochemical assays, radioimmunoassay, colorimetric analysis, immunoradiometric assays, positron emission tomography, Northern blotting, fluorometric assays, SAGE, ion-intensity based label free quantitative proteomics (LFQP), surface enhanced laser desorption/ionization (SELDI), SELDI-MS, SELDI-TOF, SELDI-TOF-MS, slot blot assay, multi-polar resonance spectroscopy, gas phase ion spectrometry, atomic force microscopy, mass-spectrometry (MS), CD, immunoassays, peptide sequencing, SDS-polyacrylamide gel electrophoresis (SDS-PAGE), electron spray mass spectroscopy, NMR, sedimentation equilibrium, flow cytometry, tandem mass spectrometry, FRET, liquid crystal-MS (LC-MS), MALDI, MALDI-TOV, MALDI-MS, microassays, ion-exchange, reverse phase HPLC, peptide mass fingerprinting (PMF), 2-D DIGE, microscale solution isoelectrofocusing (MicroSol IEF) fluorescence activated cell sorter staining of permeabilized cells, radioimmunosorbent assays, real-time PCR, hybridization assays, sandwich immunoassays, differential amplification, or electronic analysis. See, for example, Ausubel et al, eds. (2002) Current Protocols in Molecular Biology, Wiley-Interscience, New York, N.Y.; Coligan et al (2002) Current Protocols in Protein Science, Wiley-Interscience, New York, N.Y.; Sun et al. (2001) Gene Ther. 8:1572-1579; de Jager et al. (2003). Clin. & Diag. Lab. Immun. 10:133-139; U.S. Pat. Nos. 6,489,4555; 6,551,784; 6,607,879; 4,981,783; and 5,569,584; McMaster 2005, LCMS a Practical User's Guide, Wiley Interscience; McMaster, 2008, GCMS a Practical User's Guide, Wiley Interscience; Ham, 2008 Even Electron Mass Spectrometry with Biomolecule Applications, Wiley Interscience, Eidhammer et al (2008) Computational Methods for Mass Spectrometry Proteomics, Wiley Interscience; Yan & Chen, 2005, Brief Funct Genomic Proteomics 4:27-38; Zhang et al 2006 J. Proteome Res 5:2909-2918; Wang et al 2006 J. Proteome Res; Ono et al 2006 Mol Cell Proteomics 5:1338-1347; Ausubel et al, eds. (2002) Current Protocols in Molecular Biology, Wiley-Interscience, New York, N.Y.; Coligan et al (2002) Current Protocols in Protein Science, Wiley-Interscience, New York, N.Y.; and Sun et al. (2001) Gene Ther. 8:1572-1579.

Methods of characterizing a lymphoma related disorder in a subject are provided. Classifications of lymphoma related disorders include but are not limited to, a lymphoma, a lymphoma described elsewhere herein, a leukemia, and a leukemia described elsewhere herein. Therapeutic regimens or courses of treatment for lymphoma related disorders often involve medical responses with a high occurrence of deleterious side effects such as but not limited to, chemotherapy, radiation therapy, or high risk medical responses such as bone marrow transplants and transfusion regimens. Appropriate classification of a lymphoma related disorder is a significant determinant of the therapeutic efficacy of a course of treatment. Characterizing the classification of a lymphoma related disorder in a subject involves categorizing or assigning the lymphoma related disorder of a subject to a particular classification of lymphoma related disorders.

“Course of treatment” is intended to encompass a range of medical responses including but not limited to, administering one or more compounds, particularly pharmacological agents, chemotherapies, radiation therapies, surgeries, transplants, and transfusions. A disorder preferred course of treatment is a course of treatment that targets, addresses, ameliorates, improves, changes, betters, eases, controls, moderates, or regulates a sign, symptom or cause of a particular disorder. It is recognized that individual components of a course of treatment for a particular preferred disorder may also be utilized for a non-preferred disorder and that such individual components of a course of treatment for a particular preferred disorder may be administered at different dosages, ranges, concentrations, or treatment regimens for a non-preferred disorder.

A “lymphoma preferred” course of treatment is a course of treatment that targets a symptom, sign, or cause of one or more types of lymphoma. Lymphoma preferred courses of treatment are readily known to one skilled in the art. Lymphoma preferred courses of treatment may include, but are not limited to chemotherapy, radiotherapy, combination chemotherapy regimens, autologous transplantation of bone marrow, autologous peripheral cell product transplantation, stem cell transplantation, consolidation myeloablative therapy, regional radiotherapy, hydration, alkalinization, electron beam radiotherapy, sunlight, administering compounds including but not limited to mechloethamin, vincristine, procarbazine, prednisone, MOPP, doxorubicin, bleomycin, vinblastine, dacarbazine, ABVD, nitrosoureas, ifosamide, cisplatin, carboplatin, and etoposide, single alkylating drugs, two drug regimens, three drug regimens, interferon, biological response modifiers, radiolabeled antibody therapy, CHOP, cyclophosphamide, doxorubicin, CODOX-M/IVAC, cyclophosamide, methotrexate, ifosfamide, etoposide, cytarabine, IL-2, allopurinol, topical corticosteroids, adenosine deaminase inhibitors, fludarabine, 2-chlorodeoxyadenosine, folic acid antagonists, and topical nitrogen mustard. See for example Beers et al Eds. The Merck Manual of Diagnosis and Therapy, 18^{th} Edition, 2006, Merck.

A “leukemia preferred” course of treatment is a course of treatment that targets a symptom, sign, or cause of one or more types of leukemia. Leukemia preferred courses of treatment are readily known to one skilled in the art. Leukemia preferred courses of treatment may include, but are not limited to, administering platelets, packed red blood cell transfusions, transfusing granulocytes, monitoring hydration, monitoring electrolytes, monitoring urine alkalinization, irradiation, cranial nerve irradiation, whole brain irradiation, bone marrow transplantation, chemotherapy, radiotherapy, CNS prophylaxis, γ-globulin infusions, local irradiation, total body irradiation, cytokine therapy, cytoreductive chemotherapy, and administering compounds including but not limited to broad-spectrum bactericidal antibiotics, TMP-SMX, tremethoprim-sulfamethooxazole, amphotericin, acyclovir, allopurinol, multidrug regimens, prednisone, vincristine, anthracycline, asparaginase, cytarabine, etoposide, cyclophosphamide, methotrexate, leucovorin rescue, corticosteroids, mercaptopurine, daunorubicin, idarubicin, 6-thioguanine, etoposide, all-trans-retinoic acid, corticosteroids, fludarabine, interferon-α, deoxycoformycin, 2-chlorodeoxyadenosine, hydroxyurea, myelosuppressive drugs, 6-mercaptopurine, melphalan, and cyclophosphamide. See for example Beers et al Eds. The Merck Manual of Diagnosis and Therapy, 18^{th} Edition, 2006, Merck.

The term “administering” is used in its broadest sense and includes any method of introducing a medical response to a subject including but not limited to, introducing a compound into a subject. This includes directly administering a medical response, including but not limited to, introducing a compound, and indirectly administering a medical response, including but not limited to, introducing a compound. Further examples of indirect administration include but are not limited to instances in which a medical professional may direct, advise, counsel, order, or instruct another member of the medical profession, a member of the medically related arts, an affiliate thereof, a subject, a subject's caretaker or a subject's care-provider to administer a medical response including but not limited to administering compound to a subject. Methods of administering a compound include, but are not limited to, intravenous, intramuscular, oral, intraperitoneal, surgical, transmucosal, and transdermal administration.

Methods of the present application relate to optimizing therapeutic efficacy associated with treatment of a lymphoma related disorder in a subject at risk for a lymphoma related disorder. The methods are particularly useful for characterizing a lymphoma related disorder in a subject at risk for a lymphoma related disorder as a lymphoma or leukemia. As used herein, the phrase “optimizing therapeutic efficacy associated with treatment of a lymphoma related disorder” refers to adjusting the course of treatment such that administering a lymphoma preferred course of treatment is correlated with a subject at risk for a lymphoma and administering a leukemia preferred course of treatment is correlated with a subject at risk for a leukemia. Therapeutic efficacy generally is indicated by alleviation of one or more signs or symptoms associated with the disorder being addressed, an amelioration of an adverse sign or symptom associated with a compound of interest administered to a subject, or an alleviation of one or more signs or symptoms associated with the disorder being addressed and an amelioration of an adverse sign or symptom associated with a compound of interest administered to a subject. Therapeutic efficacy can be readily determined by one skilled in the art as the alleviation of one or more signs or symptoms of the disorder being addressed or an amelioration of an adverse sign or symptom associated with a compound of interest administered to a subject.

A correlative multi-level terrain visualization technique is disclosed along with some results showing biomarker discoveries for selected diseases using the technique. The visualization technique integrates biological network information of molecules and diseases as “protein terrains” and “disease terrains.” Protein-to-disease visual analytic tasks can be completed by building and analyzing a protein terrain, with a protein-protein interaction network as the base network and each protein's association strength to a given disease as the response variable of the surface rendering. Disease-to-protein visual analytic tasks can be completed by building and analyzing a disease terrain, with a disease association network as the base network and each disease's association strength to a given protein as the response variable of the surface rendering. The correlative and iterative analysis of proteins and diseases on these two terrains can enable the study cancer candidate biomarker protein-protein interaction network and cancer disease association networks together. Protein terrains or disease terrains can be robust against data noises common in biological networks.

Terrains can be used as a framework for large-scale network visualization and visual exploration. A scalar field can be rendered as a terrain surface by encoding a numerical attribute of nodes in the network and encoding connectivity among nodes as a neighborhood. Smooth terrain surfaces can be generated using an interpolation scheme to produce a continuous scalar field from scatter data. The design of a foundation layout and interpolation of scatter data both incorporate attributes of the nodes in the networks. Multi-scale visualization and other interactive schemes combined with terrain surface visualization can be used to overcome difficulties in visualizing large scale graphs. The disclosed framework arranges the expression values on a native bio-molecular base network by rendering terrain surfaces and contours upon the layout of the network, and therefore can provide rich visual and semantic information to help researchers with biomarker discovery tasks and clinicians with molecular diagnostics tasks. The disclosed framework can provide an overview of a network context in a node centric way capturing the change of the network by demonstrating the formation of landmarks, such as peaks and valleys.

The disclosed system can take advantage of the perception capabilities of human beings to detect changes in bio-molecular expression profiles as landmark features. Biologists can be benefited from the visual feedback on the profiles. Multiple exemplary embodiments are disclosed, as well as the application of the system to several disease biology studies. The principle and framework of the disclosed system can be generalized by those of skill in the art for biomarker discovery data explorations far beyond the case study examples disclosed herein. In fact, other biological ontology networks, including disease networks, pathway networks, and their dynamics can also be visualized and explored using the disclosed framework and system, given the appropriate goals of investigation and the definitions of vertices and their relationships in the networks. By adjusting and enhancing the interactivity of the disclosed framework and system, the visualization framework can further be incorporated into knowledge discovery processes in the biological domain.

The disclosed computational biomarker discovery paradigm enables biomedical researchers to iteratively and visually integrate, explore, filter, and validate biomedical domain knowledge for a specific biomarker application. This paradigm can use different types of three-dimensional terrain visualization panels that represent domain-specific network biology knowledge at two scales, for example a Molecular Network Terrain and a Phenotypic Network Terrain. Molecular Network Terrains represent modifications or changes of multiple molecular measurements organized at the molecular interaction network level. Phenotypic Network Terrains represent applicability of candidate biomarker(s) to a set of similar phenotypes organized at the phenotypic association network level.

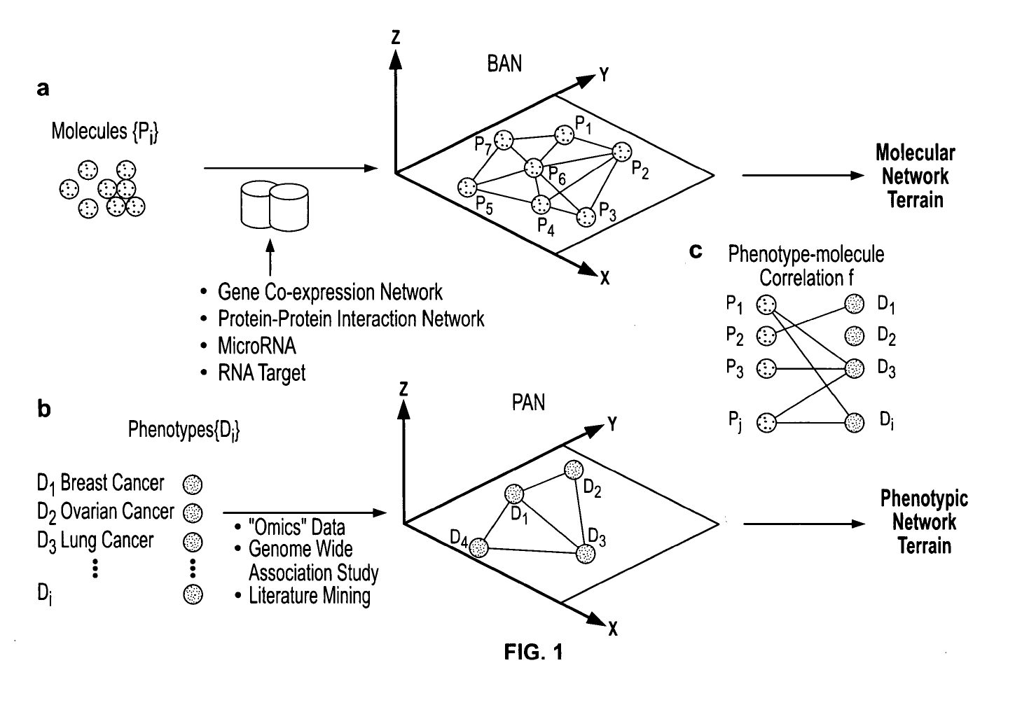

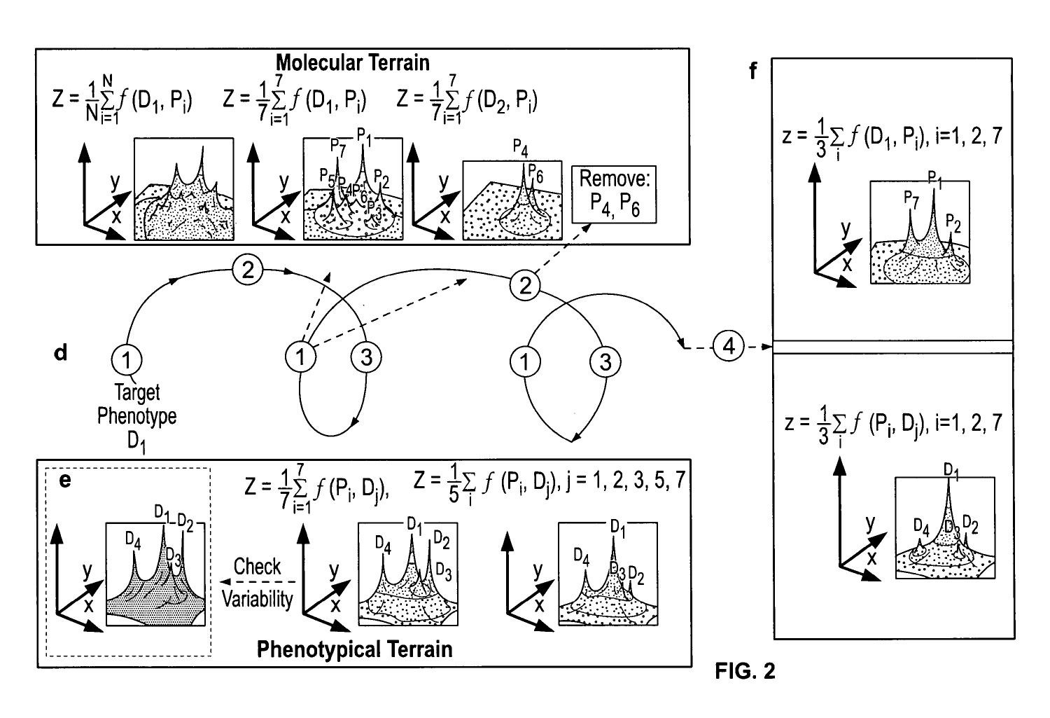

An exemplary overview of the technique using a Molecular Network Terrain and a Phenotypic Network Terrain is illustrated in FIGS. 1 and 2. Three-dimensional terrain visualization panels capture both topological information that represents associative relationships between molecules or phenotypes at its terrain base (on the x-y plane) and quantitative response variables with values interoperated over a smooth surface (on the z-axis). The area of influence for each base network node on the terrain panels can be defined, using a weight score that represents its functional properties or network topological properties in the base network. Color intensity or texture on these terrain panels may further be used to represent additional essential biomarker attributes, such as molecular measurement variability on molecular network terrain or disease prevalence on a phenotypic network terrain. Three-dimensional visual analytic tools can encourage users to take advantage of their visual perceptive strength in spatial orientation and landscape recognition, and can help users discover non-obvious relationships that are difficult to extract a priori with statistical or algorithmic techniques in complex data sets.

The method can include constructing both phenotype-specific molecular network terrains and molecular-specific phenotypic association terrains as shown in FIGS. 1a-c. The terrains are based on prior knowledge derived from literature curation, literature mining, and experimental measurements from biomarker assays. Each terrain renders a smooth surface upon a base network by interpolating quantitative measurement of each base network node as the response variable.

FIG. 1a illustrates construction of an exemplary molecular network terrain. A molecular network terrain organizes at least three types of information. A comprehensive list is collected of candidate biomarkers for a specific phenotypic context. A molecular interaction subnetwork is formed on the x-y plane among the candidate biomarkers constructed with physical molecular interactions or functional molecular associations, and a set of normalized molecular measurements is derived from available assays for each candidate biomarker in the terrain. For the example shown in FIG. 1a, the base network of the molecular terrain is a molecular interaction subnetwork and the response variable is a phenotype-molecular correlation score. The molecular terrain surface can be constructed by interpolating the response variable of each node of the base network as a height scalar.

FIG. 1b illustrates construction of an exemplary phenotypic network terrain. A phenotypic association terrain also organizes at least three types of information. A set of similar phenotypic conditions subject to biomarker specificity studies is collected. A phenotypic association subnetwork is formed on the x-y plane among related phenotypic conditions constructed with gene-sharing disease-to-disease association relationships, and a measurement of a biomarker or a biomarker panel tested for each phenotypic condition in the terrain. For the example shown in FIG. 1b, the phenotypic terrain is built with a phenotype association network as the base network, and a phenotype-molecule correlation score as the response variable. The phenotypic terrain surface can be constructed by interpolating the response variable of each node of the base network as a height scalar.

FIG. 1c illustrates that a phenotype-molecule correlation score is derived for every pair of a phenotype and a molecule forming the nodes in the molecular network terrain and the phenotypic network terrain. These correlation scores can be derived from literature mining.

FIG. 2 illustrates how researchers can analyze multiple terrain visualization panels to identify and assess candidate biomarkers. The initial identification of candidate biomarker(s) can be performed by selecting regions of high peaks or valleys in the molecular network terrain. The sensitivity of identified candidate biomarker(s) can be assessed by evaluating the height of the selected peaks or valleys relative to the molecular network terrain surface—the higher the more sensitive. The disease specificity of selected candidate biomarker(s) can be assessed by evaluating the height of selected peaks relative to phenotypic network terrain surface—the higher the more specific. Additionally, the variability of measured biomarker(s) can also be assessed, by evaluating color-intensive surface of the molecular network terrains, if such information is represented.

To develop biomarker panels with satisfactory sensitivity and specificity using the disclosed framework, a four-step iterative refinement process of biomarker development using terrain visualization panels can be followed. FIG. 2 illustrates this process for phenotype D₁, to achieve a high quality molecular biomarker panel with satisfying disease sensitivity and specificity. Step 1 is the composition of a biomarker panel. Step 2 is the removal of poor biomarker(s) from the panel. Step 3 is a sensitivity and specificity assessment of the biomarker performance. Step 4 is finalization of the biomarker panel. Steps 1-3 may be iterated multiple times until a desirable biomarker panel with satisfying performance is found. Optional steps can be added, for example to check the variability of the current molecular biomarker panel's variability. Color coding can map the variance of the correlation scores between biomarkers in the panel and phenotypes. The achieved molecular terrain of the candidate biomarker panel after the fourth step (far right of FIG. 2) shows a satisfying sensitivity visual pattern, and the achieved phenotypical terrain shows a satisfying specificity visual pattern of the panel.

While molecular network terrain alone can be used to identify initial candidate biomarkers for a specific disease, the disease specificity is revealed on the corresponding phenotypic network terrain. Factors such as the quality and coverage of molecular interaction/association networks can affect the shape and characteristic peaks of terrains. However, varying quality and coverage of human molecular interaction/association data has much more impact on the contour of molecular network terrains built for the dissimilar diseases than those built for the same or similar diseases. Overall, terrain features such as major landscape, characteristic peaks, topological relationships among major peaks are relatively stable, suggesting they are robust against noise derived from different network construction methods.

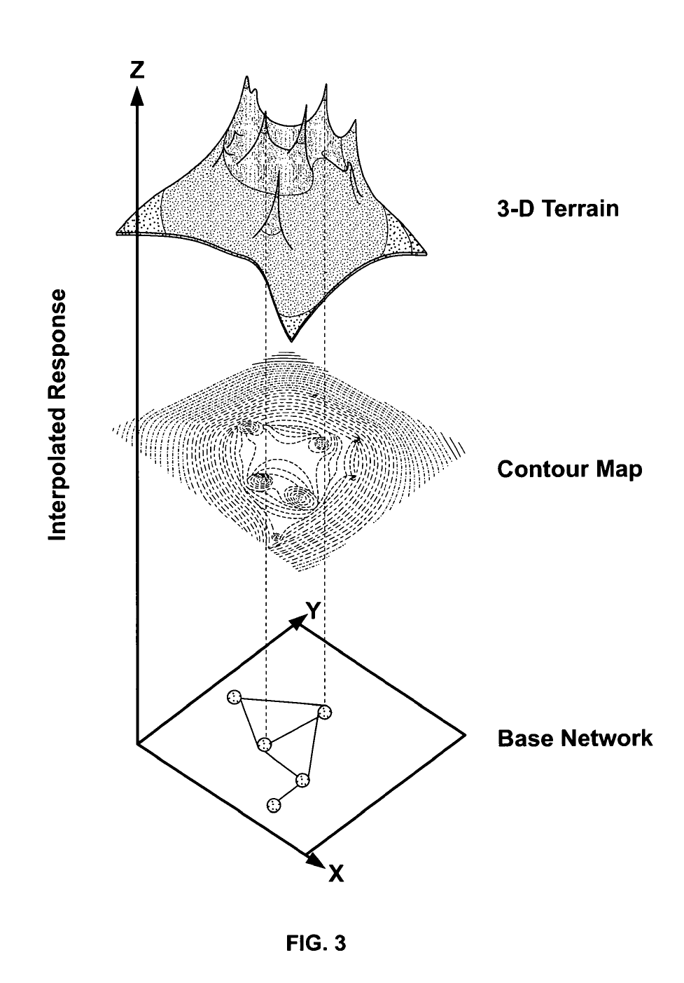

More detail of the terrain construction process will now be described. FIG. 3 shows a three-dimensional terrain derived from a two-dimensional base network in the x-y plane and a response variable for the z-coordinate. An interpolated smooth terrain surface can be built on the base network by interpolating values of the response variable (z-coordinate) on each node point of the base network. A contour map is a cross section of a terrain at a specific response variable value (height).

The base network of a terrain can be represented by a general node-weighted, edge-weighted undirected graph as:

Gᵢ={V,E,f,g,O,C}, where

V is the set of nodes,

E is the set of edges,

f assigns a weight value to each node, f: V→R,

g assigns a score to each edge, g:E→R,

O is the center position of the planar graph in world coordinates, and

C is the scale of the graph.

The grid scale for the base map of terrain rendering can be defined based on C.

An adapted node-weighted-and-edge-weighted spring embedder graph drawing algorithm can be used to generate the graph node layouts in the base network. This spring embedder graph drawing algorithm can work as follows: if an edge connects a pair of nodes then the resting distance of the spring connecting the pair of nodes is inversely proportional the edge score; otherwise, the resting distance of the spring connecting the pair of nodes is proportional to the summation of the node weights, which defines an area of influence for each node. Different from conventional spring embedder graph drawing algorithms, this method separates hub nodes in the graphs.

In the base network layout, nodes in the original networks can be laid out in two steps: initial layout and optimization. Though the layout algorithm gives priority to nodes with larger weights, it also keeps them compact. Drastically differing distances among pairs of nodes can cause the resolution of grids to be arbitrarily small, which can in turn lead to aliasing problems in rendering. Intuitively, nodes with larger weights push other nodes aside while edges pull end nodes closer. The final position of each node is the accumulated effect of the constraints imposed on it. The node and edge functions, f and g, are used to quantify the constraints. The improved layout of the graph is achieved by optimizing this constraints-based system.

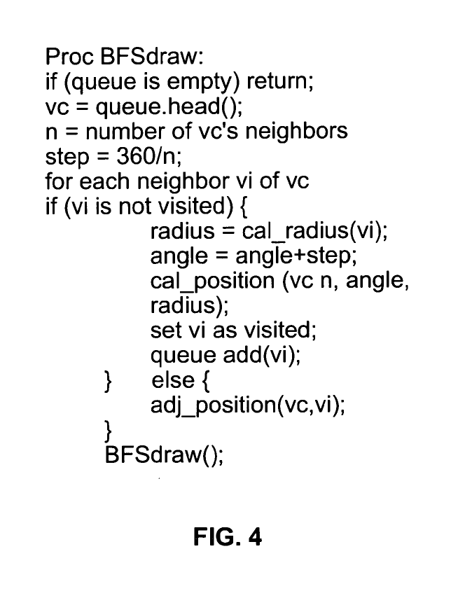

In the initial layout, the graph can be configured manually to approximate the global minimum before the optimization, in order to avoid local minima in the process of optimization. The nodes can be arranged in two-dimensions and kept planar during the optimization. Each node vᵢ, with f(vᵢ) larger than threshold T_{f} is radially laid out around point O. The radius can be proportional to log(f(vᵢ)) which reflects the idea that nodes with larger weight push each other aside. A logarithmic scale can be used here and later in the model to reduce any significant difference of distance among pairs of nodes. Starting from one of those nodes, an extended version of Breadth First Search (BFS) can be carried out to determine the position of other nodes. The node can be radially laid out around its parent when it is first visited, and the position can be adjusted each time it is revisited by other nodes. The algorithm can be outlined by the pseudo-code shown in FIG. 4, where:
cal_radius( ) calculates the radius of v_{C} for the radial layout around v_{C} depending on g(vᵢ, v_{C}), f(vᵢ), and f(v_{C}),
cal_position( ) calculates the actual position for vᵢ, and
adj_position( ) adjusts vᵢ's position depending on g(vᵢ,v_{c}), f(vᵢ), and f(v_{c}).
   The actual algorithms of cal_position( ) and adj_position( ) can be designed similar to the energy minimization model discussed below.

To optimize the constraints-based system, the spring embedder (force-direct) model can be applied. The classical spring model is:

$$E = \frac{1}{2} {\sum }_{i \neq i}^{ } {{k}_{ij} ( \vert p ( {v}_{i} ) - p ( {v}_{j} ) \vert - {l}_{ij} )}^{2} ,$$

where

p(vᵢ) is the position of node vᵢ;
lᵢⱼ is the ideal spring length for node vᵢ and vⱼ, which is usually a predefined path between the two nodes, and
kᵢⱼ is the Hook coefficient.
   This model can be generalized as a multi-dimensional scaling model, where |p(vᵢ)−p(vⱼ)| is the original distance of the two nodes in d dimension and lᵢⱼ is the distance in projected d′ dimension (d≧d′). Each of the terms in the general model is redefined based on constraints. Note that weight f and interaction strength of an edge g are two important factors. In addition, there are two types of constraints for placing the node pairs (vᵢ, vⱼ): node constraints and edge constraints.

Node constraints are used to position nodes together to keep the layout compact. Each node has an area of influence which is a circular area with the node at the center. When a pair of nodes does not have any edges between them, the nodes tend to push other nodes out of their area of influence. In other words, two areas of influence tend not to overlap under this circumstance. The radius of the area of influence is determined by f(vᵢ) and f(vⱼ). Edge constraints tend to pull two nodes connected by an edge closer together. The area of influence can somewhat overlap, however, the distance between the centers of the two areas of influence is still preserved by g(vᵢ, vⱼ). Node and edge constraints will influence the final position of node pair (vᵢ, vⱼ). Pairs of nodes having no edges between them are subject to node constraints, whereas pairs of nodes having edges between them are subject to edge constraints. Therefore, the force-direct model can be characterized by:

$$E = \frac{1}{2} \text{?} {( \vert p ( {v}_{i} ) - p ( {v}_{j} ) \vert - \text{?} + f \text{?} )}^{2} + {\sum }_{( {v}_{i} , {v}_{j} ) ∈ E}^{ } ( \vert p ( {v}_{i} ) - p ( {v}_{j} ) \vert - g {\text{?}}^{2} ) . \\ \text{?} \text{indicates text missing or illegible when filed}$$

where log(f(vᵢ)+f(vⱼ)) is the ideal projected distance for nodes vᵢ and vⱼ when they do not have edges and g(vᵢ, vⱼ) is the ideal projected distance for nodes vᵢ and vⱼ when they share an edge. Nonlinear system minimization techniques can be applied to minimize the energy of this model. Conjugate gradient can be used to estimate the descent direction in N dimensions.

As defined above, O is the center and C is the scale of the graph. The optimized layout can be scaled to fit into a bounding square that centers at O and has edge length C. The grids can be defined to be the same size as the bounding square that centers at O as well. If the shortest distance between any pair of nodes is βC after minimization, where β<1, the resolution of the grids can be defined to be smaller than βC, so that no cell of the grid has more than one node.

At this point, the grid containing the optimized two-dimensional base network layout is ready for surface rendering. Suppose the value of a terrain's response variable vᵣ is f(v_{b}, vᵣ) for each node v_{b} in the base network, then the response value is treated as the vertical elevation for v_{b} in the z dimension. The final terrain surface includes points elevated from the base network at the nodes, and interpolated points between these elevated points. The interpolated points can be computed using the Sherpard displacement interpolation method. The response variable can represent any other additional attribute of the node, or can be computed from the functional mapping of multiple underlying variables. A terrain computed from the functional mapping of multiple underlying variables can be referred to as a consensus terrain. For a consensus terrain, a linear equal-weighted function can be used to combine the response variables for a node such that the vertical elevation of each point ρ in the consensus terrain is calculated as the average elevation of individual response variables. The response variables are then rendered as elevations to generate a height field from the two-dimensional base network plane where the nodes reside.

Sherpard's method, originally proposed in 1968, is one of the simplest interpolation techniques. It takes the distance weighted average of the interpolation points as the interpolation function. An improved Sherpard's method was proposed later, which interpolates the displacements of the points. In our scattered data interpolation, a scalar value is used as “displacement.” Therefore, the unknown scalar value for each grid point can be computed by:

$$s ( p ) = {\sum }_{i = 1}^{n} \frac{s ( {v}_{i} )}{\text{?}} / {\sum }_{i = 1}^{n} \frac{1}{\text{?}} . \\ \text{?} \text{indicates text missing or illegible when filed}$$

where

p is the grid point with unknown scalar value,

s(vᵢ) is the scalar value of node vᵢ,

d′ᵢ(p) is the distance from node vᵢ to p, and

r is the exponent parameter to weigh the factor of distance.

Using area of influence, nodes with different weight f(vᵢ) are not interpolated as they are symmetric points in interpolation. The scalar value of nodes with larger weights should have more influence on the scalar value of the grids than nodes with smaller weights. Thus, the modified Sherpard's method is as follows:

$$s ( p ) = {\sum }_{i = 1}^{n} \frac{s ( {v}_{i} ) * f ( {v}_{i} )}{\text{?}} / {\sum }_{i = 1}^{n} \frac{f ( {v}_{i} )}{\text{?}} , \\ \text{?} \text{indicates text missing or illegible when filed}$$

where f(vᵢ) is the weight factor in interpolation.

The scalar value of each grid point is rendered as an elevation from the two-dimensional plane of the foundation or base network layout. The position of the elevated point q of grid point p(x, y) is (x, y, α*s(q)), where α is a uniform scale factor. The height field can then be rendered as a surface, given that the scalar values of the grids points are available. The visualization display software can be used to generate the terrain surfaces and contours based on the height values. A color scheme can be adopted to denote different heights. Let α*s(vᵢ) be H(vᵢ). If H(vᵢ) is larger than a certain value Sᵢ, then vᵢ in the two-dimensional plane of contour rendering will be enclosed by the contour of value Sᵢ.

A visualization paradigm is disclosed that investigates the relationships among correlative multi-level graphs of interacting biologically entities. The links of correlative multi-level graph can be derived from association mining of a biomedical literature collection. The visual paradigm can represent this multi-level graph in multiple components. A terrain surface visualization includes a base network and a response variable as a node attribute in the network. One or more biological entities can be treated as the response variable to render a terrain surface on top of the nodes. A pair of networks can be correlated in the multi-level graph by rendering the terrain surface as nodes in one of the networks, using the other network as the base network. This paradigm can be applied to a pair of networks, for example a correlative core cancer term network and a core gene term network. The visualization paradigm is consistent with the derived associations, and effectively preserves the major features in the correlations among entities.

To show the construction and usage of the visualization paradigm, a sample data set can be created of a cancer term network and a gene term network, and the interactions between any two entities in the two networks can be quantified by associations between the two corresponding terms.

Different types of cancers and their related genes, for example cancer causing genes and biomarker genes, are of prime interest in current biological and pharmaceutical discoveries. Translational association literature mining can be used to collect data on the cancers and related genes. For cancer terms, 244 unique cancer terms from MeSH are included in this example. The gene terms are then retrieved by using cancer terms to query the PubMed abstracts collection. For every query pass, only a constant number of returned gene terms are kept (in this example, the constant number is 20), and subsequently, 768 unique gene terms are retrieved. The Uniprot naming convention was used to label each gene. Also, during the querying process, the top 20% of all article abstracts returned were kept for later mining. Finally 37487 unique abstracts were kept in the document collection.

The associations between any two terms aₚ and a_{q} can be calculated by the method proposed for transassociations mining, which factors in both co-occurrences in the abstracts collection and the indirect associations inferred by transitive closures. The following is a summary of this exemplary method:
Step 1. Calculate the weight of term aₖ in one document i, Wᵢₖ, using the tf-idf algorithm.
Step 2. Identify the score of co-occurrences between any two terms aₖ and aₗ, by summing up their weight in each document i.

associations[k][l]=Σᵢ₌₁^{N}Wᵢₖ+Wᵢₗ,k=1,2 . . . m,l=1,2, . . . m
Step 3. Identify the indirect association between any two terms, assuming that a transitive relation R could apply onto the terms associations:

∀aₚaᵣa_{q},(R(aₚ,aᵣ),R(aᵣ,a_{q}))→R(aₚ,a_{q})
where aₚ, aᵣ, and a_{q} are terms. We first obtain a binary matrix A for the co-occurrences of all such pair of terms in association. Then a transitive closure A* of the binary matrix is computed. In TA=A*−A, each non zero TA(i,j) indicates the existence of an indirect association between the two terms.
Step 4. Score the associations between two terms. In each non zero cell TA(i,j), identify the segments of the paths, and look up the score of each segment in associations calculated before. The score of such a path is the summation of the segment scores. The score of association between terms is the minimum among the scores of all paths.

The three-dimensional terrain surface as described above is constructed from a two-dimensional base network in the x-y plane and a response variable in the z-direction. A terrain is rendered with a smooth surface by interpolating values of the response variable for each node point of the base network.

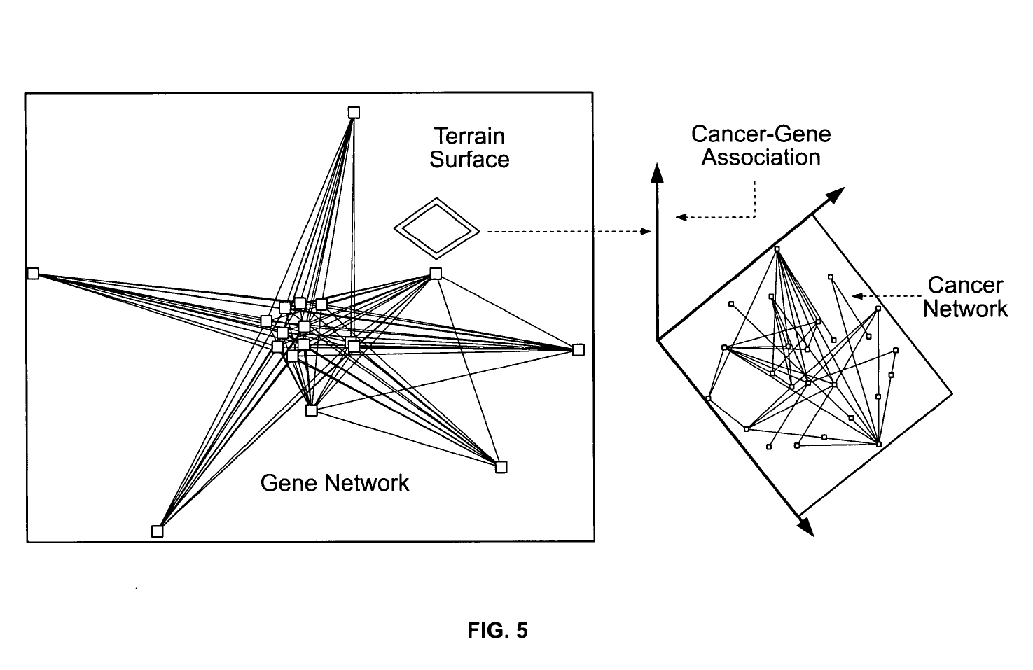

The response variable in the terrain surfaces of this exemplary study represents one biological entity (e.g. a cancer term), and the base network can reference to one network in the multi-level graph (e.g. a gene term network). The response variable values hence are the association values between the cancer term and a gene term. The arrangement puts terrain surfaces on top of the nodes, which can be laid out by multi-dimensional scaling with the distance between any nodes proportional to their association values. For instance, FIG. 5(a) shows a schematic arrangement of a terrain surface on top of a node in a cancer term network, and FIG. 5(b) shows the formation of the terrain surface in FIG. 5(a) with a gene term network as the base network. As the scale of the network in FIG. 5(a) increases, only limited space is available. So in the arrangement, based on the resolution, entity nodes can be clustered to render their consensus terrain surface as a summary and the consensus terrain surface can be put in the centroid of the cluster.

In the multi-level graph, the connections between any two graphs are important to have an understanding beyond a network of entities belonging to the same category (e.g. cancer term). Therefore, in the visual paradigm, the connections between two inter-connected networks can be represented via correlating the arrangements of the terrain surfaces on top of the two networks. For instance, to correlate the inter-connected cancer term network and the gene term network, the same gene network can be used as the base network for terrain surfaces in the cancer term network, and the cancer term network can be used as the base network for the terrain surfaces in the gene term network, and the response variable values can be from the cancer-gene term associations calculated above.

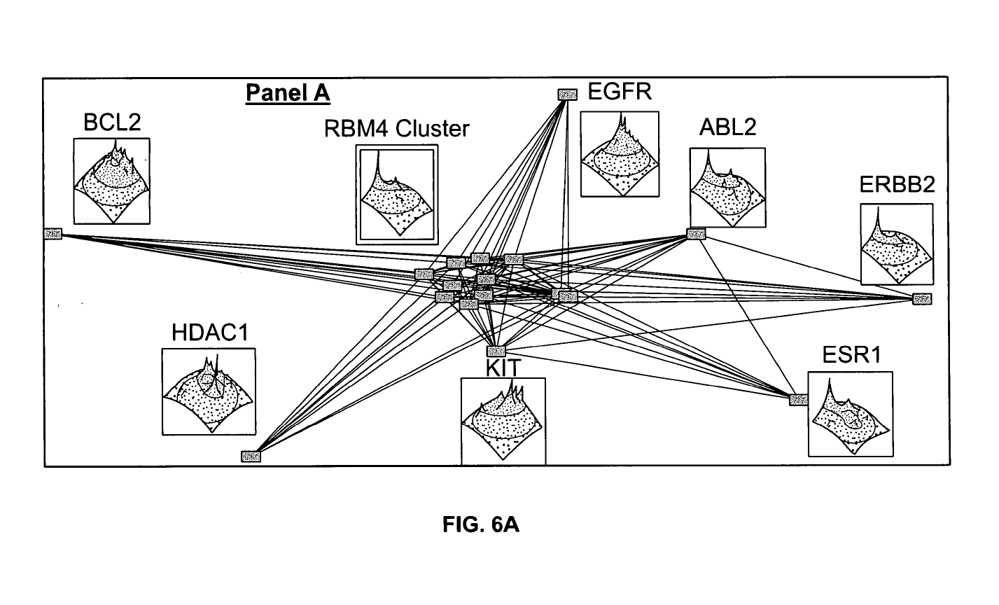

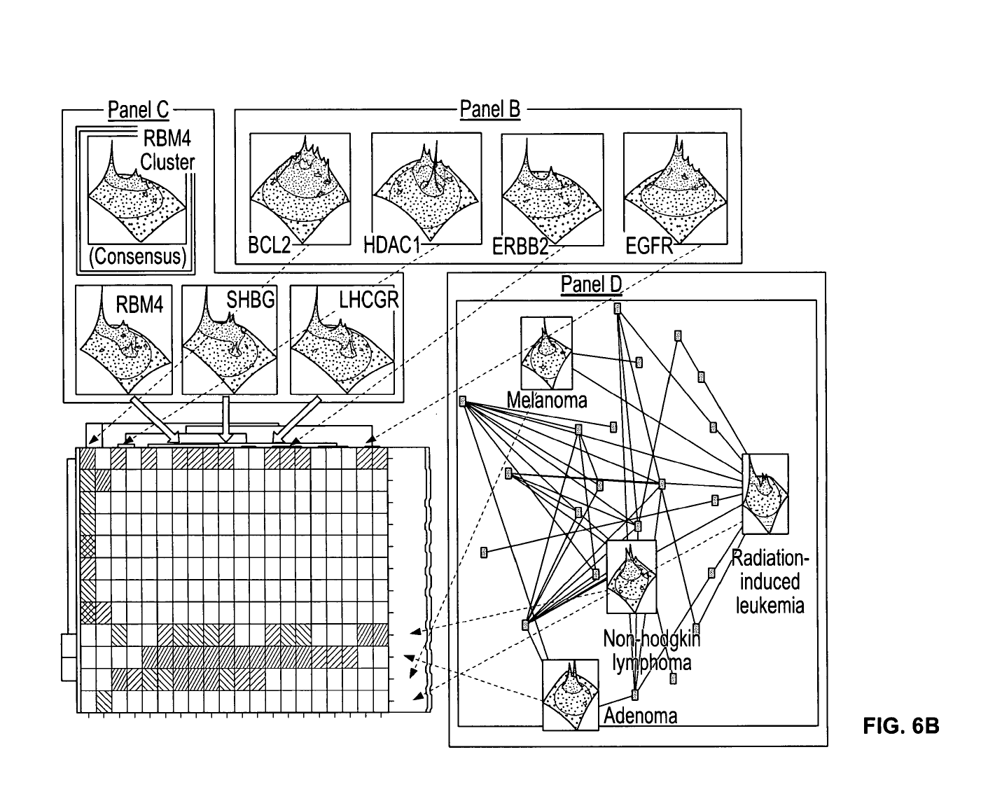

To extract the cancer-gene relation for this exemplary case, the information of the core cancers and relevant genes was further distilled from the multi-level graph data set. The twenty-five cancer terms representing the top killing cancers were identified and chosen for the connected subnetwork of twenty-five terms as the core cancer network. A connected subnetwork of twenty core cancer genes was also chosen. The core gene term network is shown in FIG. 6A. The terrains shown in FIG. 6A can be called disease terrains because the underlying base network refers to the core cancer network which is illustrated in FIG. 6 Panel D. The terrains in FIG. 6 Panel D can be called gene terrains because their base network refers to the gene term network in FIG. 6A. FIG. 6 Panel B shows detailed views of four disease terrains shown in FIG. 6A. The four corresponding gene nodes of these disease terrains are spatially separated from each other as shown in FIG. 6A. These four terrains have significantly different shapes. FIG. 6 Panel C shows an L-shaped hierarchy of three disease terrains for the three gene nodes in ‘the RBM4 cluster.’ The ‘RBM4 cluster’ terrain is a consensus terrain generated by clustering genes ‘RBM4,’ ‘SHBG’ and ‘LHCGR’ together. These genes are clustered because the three genes are cluttered with each other in the gene core network of FIG. 6A. From observing the terrains of the RBM4 cluster, we can see that genes that are close together in the network tend to have similar terrain shapes. So the layout of the gene network is consistent with the shape variations that appear in the disease terrains of the gene nodes. The further away two genes are, the more differing shapes their terrains tend to have. Similar observations could be made from the general trend on terrain surfaces in Panel D of FIG. 6. The results of the visual diagram validate the method used to build up the multi-level graph of biology entities, as the terrain surface shape variations are consistent with the position of the node in the network.

In a disease terrain for a gene, each peak represents a strong correlation between the gene and one of the diseases in the base network. Major peaks were identified in FIG. 6A in all disease terrains in the gene network. These major peaks were recorded in a disease-gene heatmap shown in FIG. 6. In the disease-gene heatmap, each row represents a cancer and each column represents a gene. The colors represent the different scale of the peaks. A two-way clustering was performed on the heat map. In the clustering results of genes, the four gene nodes in FIG. 6 Panel B that are far away from each other and have differing terrain shapes appear to belong to four well separated clusters. In the clustering results of cancer terms, four cancers, namely ‘adenoma,’ ‘melanoma,’ ‘non-Hodgkin lymphoma’ and ‘radiation-induced leukemia’ were found to belong to four well separated clusters. After referring the four cancers in the core cancer network, the corresponding nodes were found to be spatially separated. From the results of FIG. 6, it can be concluded that the major peaks in terrains, as represented as dark cells in the heatmap, are well preserved features that could indicate how the nodes should be positioned among others. The results also show the visualization power of terrain surface visualization, as the associations are presented by landmark features in the terrains, and the insignificant peaks are filtered out by human perception. Based on the major peaks, clustering the diseases appears to yield more informative disease clusters.

Exemplary Implementations of the Visualization Technique

The base networks of phenotypic-specific molecular network terrains can be constructed from candidate cancer biomarker protein-protein interaction networks. As an example, candidate cancer biomarker proteins were taken from a literature-curated protein-interaction dataset of 1049 cancer candidate biomarkers (M. Polanski, N. Anderson, Biomarker Insights 2, 1 (2006)), which primarily includes differentially expressed proteins or genes in cancer. The source of human protein-protein interaction data are collected from the Human Annotated and Predicted Protein Interaction database (HAPPI), which is a comprehensive compilation of experimental and computationally-predicted human protein interactions primarily from the OPHID (Online Predicted Human Interaction Database) and STRING (Search Tool for the Retrieval of Interacting Genes/Proteins) databases. The reliability of protein-protein interaction information in HAPPI is quantified using H-scores ranging between 0 to 1 or a quality star rank grade of 1, 2, 3, 4 or 5. Increased protein interaction grades from 1 to 5 have been shown to be associated with improved quality of physical interacting proteins and decreased amount of non-physical interactions found primarily in text mining or gene co-expression studies. Protein interactions in the HAPPI database with star grade of 3 are comparable to the overall quality of the Human Protein Reference Database (HPRD) and include mostly physical protein interactions. HAPPI was used instead of the HPRD because of its coverage of more than 280,000 human protein interactions with a star grade of 3 and above, comparing favorably with a count of less than 40,000 for HPRD. These or other relevant databases can be used as appropriate. In the HAPPI database, 762 of 1049 cancer candidate biomarkers can be matched with the Universal Protein resource (UniProt) accession numbers. Use of the HAPPI-n base network refers to a base network generated by building a protein-protein interaction network involving only those candidate biomarker proteins that are connected by HAPPI protein interactions of quality grade n and above.

In this exemplary implementation, two classes of disease base networks were built for molecular-specific phenotypic association terrains. The first class of base network, CNG, was built from disease-gene associations reported in the Online Mendelian Inheritance of Man (OMIM) database. The CNG base network is built by connecting a pair of cancer types if they share at least one gene reported by the OMIM database. In this exemplary CNG base network, only 98 different cancer subclasses were kept of the 1284 diseases subclasses defined in the work of K. Goh et al., Proceedings of the National Academy of Sciences 104, 8685 (2007), and these were further narrowed down to 60 major cancer categories for this study. CNG was further classified into CNG-I and CNG-II, based on the minimal number of shared cancer genes reported in the OMIM database for the CNG. Therefore, CNG-I is the same as the original CNG sharing minimally one gene in common between any two cancers, whereas CNG-II is a more stringent version of CNG sharing at least two genes in common between any two cancers. For this exemplary system, CNG-I contains 39 major cancer nodes in its largest connected sub-network, whereas CNG-II contains 16 major cancer nodes in its largest connected sub-network.

The second class of base network, CNL, is built from disease-gene term co-occurrence reported in the literature. The edge score f(v_{a,1}v_{b}) between two terms vₐ and v_{b} is calculated as:

f(v_{a,1}v_{b})=ln(dfᵥ_{ₐ}_{,v}_{_{b}}*N+λ)−ln(dfᵥ_{ₐ}*dfᵥ_{_{b}}+λ) (1.1)

where dfᵥ_{ₐ} and dfᵥ_{_{b}} are the number of documents in which term vₐ or term v_{b} occurred, respectively; and dfᵥ_{ₐ}_{,v}_{_{b}} is the number of documents in which vₐ and v_{b} co-occur in the same document. N is the number of documents in all PubMed (a free database maintained by the U.S. National Library of Medicine) abstracts, λ is a small constant (λ=1 in this example) introduced to avoid out-of-bound errors. Edge score f is not considered if there is no edge between vₐ and v_{b}, which means any of dfᵥ_{ₐ}, dfᵥ_{_{b}}, or dfᵥ_{ₐ}_{,v}_{_{b}} has a value of 0. The resulting function f is positive when the co-occurrences of the pair of terms are over-represented, and negative when under-represented. In this example, each cancer-cancer association edge in CNL also carries a normalized positive score, conf, to indicate the strength of disease association relationships. Similar to the classification of CNG, CNL can also be classified into CNL-I and CNL-II, to indicate their different qualities. CNL-I contains CNL sharing two diseases with a minimal strength conf score of 1.0, whereas CNL-II contains CNL sharing two diseases with a minimal strength conf score of 2.0. Both CNL-I and CNL-II preserve 56 of the 60 major cancers.

In both types of base networks, CNG and CNL, a node weight function w is defined to measure the node's connectivity based on the conf scores of its edges.

The response variable of molecular network terrains and phenotypical network terrains in this exemplary experiment can be either protein-to-disease association strengths or disease-to-protein association strengths. The reported functions between genes and diseases in the Gene Reference Into Function (GeneRif) database were used to generate the disease-gene association matrix in this example, but other sources could also be used. A strength score is recorded in the association matrix between two associated terms—a disease represented using its Medical Subject Headings (MeSH) term and a gene (with all gene or protein synonyms)—regardless of the direction of associations identified. The proteins were taken from 762 HAPPI-overlapped cancer candidate biomarkers, whereas the diseases were taken from 56 major cancers in CNL. For each cancer-protein association, its association strength can be calculated using equation 1.1 shown above. The association strength scores can be normalized between a pair of cancer and candidate protein biomarkers, by dividing the original association strength score with the average of all association scores for the cancer involved in the normalization. Normalization helps make fair comparisons of response values across both popular and rare cancer types.

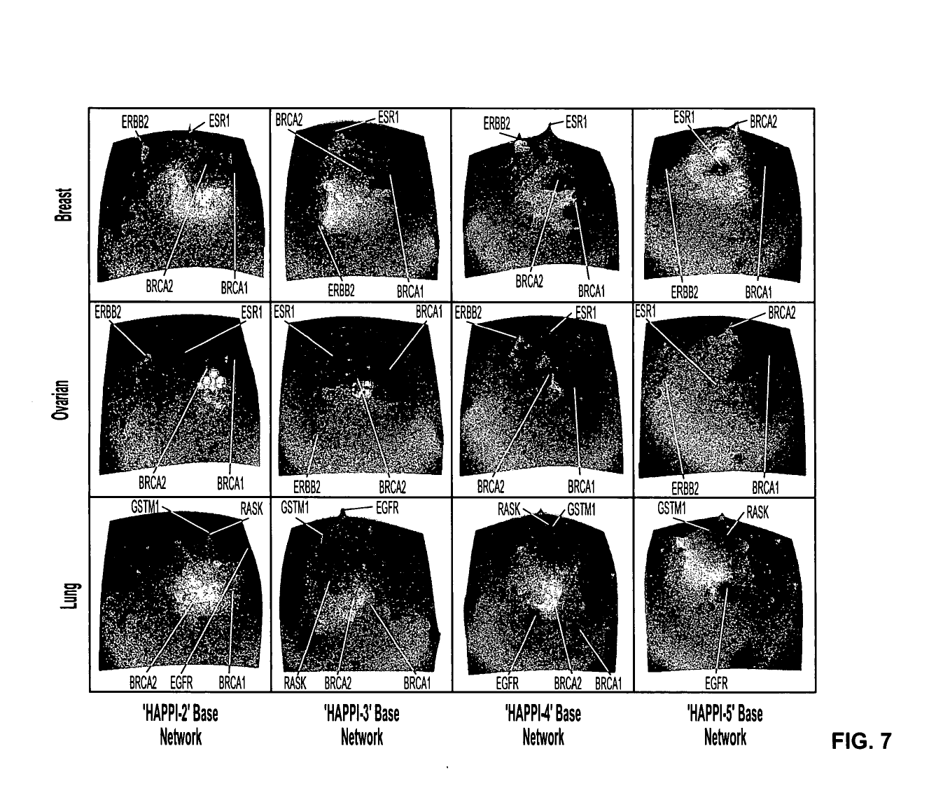

FIG. 7 shows a row of four molecular network terrains developed for each of breast cancer, ovarian cancer, and lung cancer, respectively. The protein terrains for each cancer are varied among four types of protein interaction base networks of increasing quality, HAPPI-2, HAPPI-3, HAPPI-4 and HAPPI-5, respectively. We can make many interesting observations from the protein terrains shown.

For breast cancer (first row) and ovarian cancer (second row), molecular network terrains identified candidate biomarkers are BRCA_{—}1 HUMAN (Breast cancer 1), BRCA_{—}2—HUMAN (Breast cancer 2), ESR1_HUMAN (estrogen receptor 1), and ERBB2_HUMAN (Human Epidermal growth factor receptor 2, HER₂). For lung cancer (third row), molecular network terrains identified candidate biomarkers are EGFR_HUMAN (Epidermal growth factor receptor 1), RASK_HUMAN (KRas proto-oncogene protein), GSTM1_HUMAN (Glutathione S-transferase Mu 1).

In FIG. 7, we can identify well known genetic markers for these cancers, by following any column (fixed protein interaction base network quality), e.g., for “HAPPI-5” base network, and relate major peaks to regions of gene cluster regions highly associated to any of the three cancers. Here, the heights of the major peaks suggest the sensitivity performance of a candidate biomarker, and the higher the peak rises above the surface, the more sensitive the candidate protein biomarker. For breast cancer, BRCA1, BRCA2, ESR1, and ERBB2 are four major characteristic peaks. For ovarian cancer, the same set of four proteins still dominates the protein terrain landscape. For lung cancer, EGFR, RASK, GSTM1 are characteristic peaks. Abundant literature studies can be found to confirm that BRCA1, BRCA2, HER2, and ESR1, among other genes, are major genetic markers and risk factors for breast cancer and ovarian cancer. Defects in EGFR, RASK, and GSTM1 are also strongly associated with lung cancer.

The major landscapes and peaks from these dominant genetic cancer markers do not appear to be affected by different base network layouts developed from protein interaction data of varying qualities, showing that the terrain profiles are robust against noise in the base network layouts. This can be confirmed by comparing gene terrains across different columns for the same cancer type in FIG. 7. However, subtle patterns of landscape differences on smaller peaks do exist. This could be attributed to the fact that the base network layout for higher quality cancer biomarker protein interactions contains fewer proteins (727 for HAPPI-2, 717 for HAPPI-3, 679 for HAPPI-4, and 562 for HAPPI-5) and protein interaction clusters on the protein terrain. During the surface interpolation step to generate protein terrains, regions filled with proteins with higher node weights (due to higher degree of interaction connections) could lead to higher peaks. Therefore, more details of small peaks can be observed for the breast cancer protein terrain series generated with lower interaction data qualities, while higher peak levels can be observed for the ovarian cancer protein terrain series generated with lower interaction qualities as well.

The relative distances and topological relationships of major peaks also seem to be stable, resistant to variations of interaction data quality of the base networks. For example, the BRCA1_HUMAN and BRCA2_HUMAN peaks are consistently clustered closer together than they are to any of the other protein peaks, including ESR1_HUMAN or ERBB2_HUMAN, in breast cancer and ovarian cancers.

FIG. 7 also shows that diseases that are similar to each other share more similar protein terrain landscapes than diseases that are different. Compare the protein terrains between two female cancers, breast cancer and ovarian cancer, and a female cancer against lung cancer within the same column. It is apparent that protein terrains for breast cancer and ovarian cancers not only share similar genetic markers but also similar protein terrain landscapes. This is not the case for breast cancer and lung cancer.

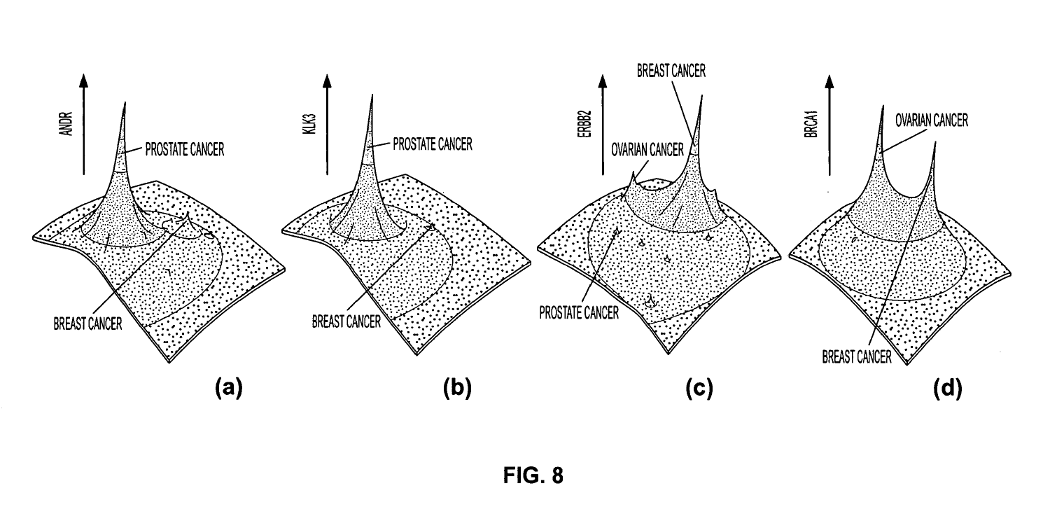

FIG. 8 shows disease terrains developed for four cancer biomarkers well-documented in the literature to examine their disease biomarker specificity. FIGS. 8(a) and 8(b) show two potential candidate biomarkers for detection of prostrate cancer, and FIGS. 8(c) and 8(d) show two potential candidate biomarkers for detection of ovarian cancer. All these disease terrains have the same base network, the cancer disease association network (type CNL II), which is derived from a method described above. Note that we made similar experimentations as we did for protein terrains by altering disease base networks to make the choice of an overall good CNL II base network. The characteristics peaks and landscape pattern in disease terrain can hypothesize and rate the disease specificity for well-documented cancer biomarkers; the higher the peak indicates the more specific the biomarker.

By comparing FIGS. 8(a) and 8(b), we can observe that ANDR_HUMAN (Androgen Receptor) and KLK3_HUMAN (Prostate specific antigen, PSA) are potential candidate biomarkers for prostate cancer, because the peaks for prostate cancer in the two disease terrains—suggesting the sensitivity performance of these two protein biomarkers for prostate cancers—are both much higher than other peaks (e.g., breast cancer as the second most visible peak). This observation is consistent with literature findings. Since the disease terrain surface for candidate biomarker PSA is cleaner than ANDR, and the second most visible peak for breast cancer is smaller, PSA appears to be a better single biomarker for prostate cancer. Also, since the disease terrains between PSA and ANDR are similar, a panel biomarker by simple aggregating these two proteins in a same assay may not be a good idea.

FIGS. 8(c) and 8(d) show the disease terrains for ovarian cancer with candidate biomarkers ERBB2_HUMAN (HER2) and BRCA1_HUMAN (BRCA1). These disease terrains for detection of ovarian cancer show results that are consistent with literature knowledge that HER2 is broadly associated with many types of cancers while BRCA1 is strongly associated with female cancers more specifically. Therefore, neither of the two proteins should be used for general-purpose cancer subtyping applications. With better specificity than HER2, however, BRCA1 could potentially be developed for distinguishing female cancers from other cancer types.

Alzheimer's Disease

Alzheimer's Disease (AD) is a progressive neurodegenerative disease diagnosed in almost five million people in the US today. The number of diagnosed AD patients is also expected to quadruple from its current number worldwide in the next forty years. The mental status of an AD patient deteriorates irreversibly over time, therefore an early diagnostic test to treat AD with high precision bears the highest hope of helping deter the onset and progression of the disease. However, there have not yet been approved AD molecular diagnostic tests with enough sensitivity and specificity.

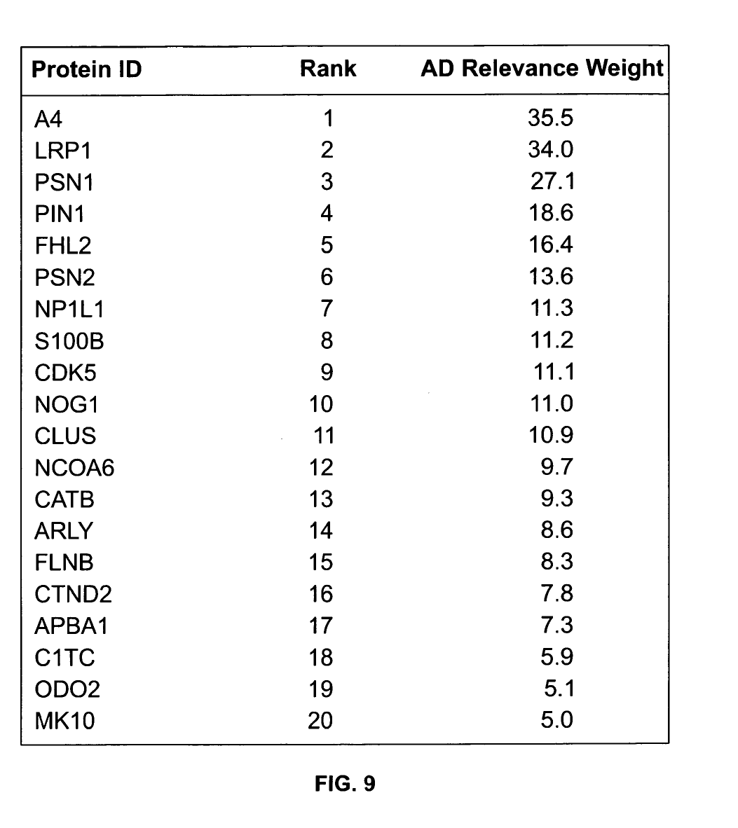

An AD protein interaction network was laid out as described above. In the AD gene terrain, edges disappear and are replaced by topological neighborhoods in the terrain. Nodes become noticeably significant, occupying an area proportionally to its relative significance, which is based on the calculated AD-relevance gene ranking score shown in FIG. 9. FIG. 9 shows the top twenty significant proteins identification and weights, and this data is derived from Chen et al., “Mining Alzheimer disease relevant proteins from integrated protein interactome data”, Pacific Symposium on Biocomputing 2006; 11: 367.

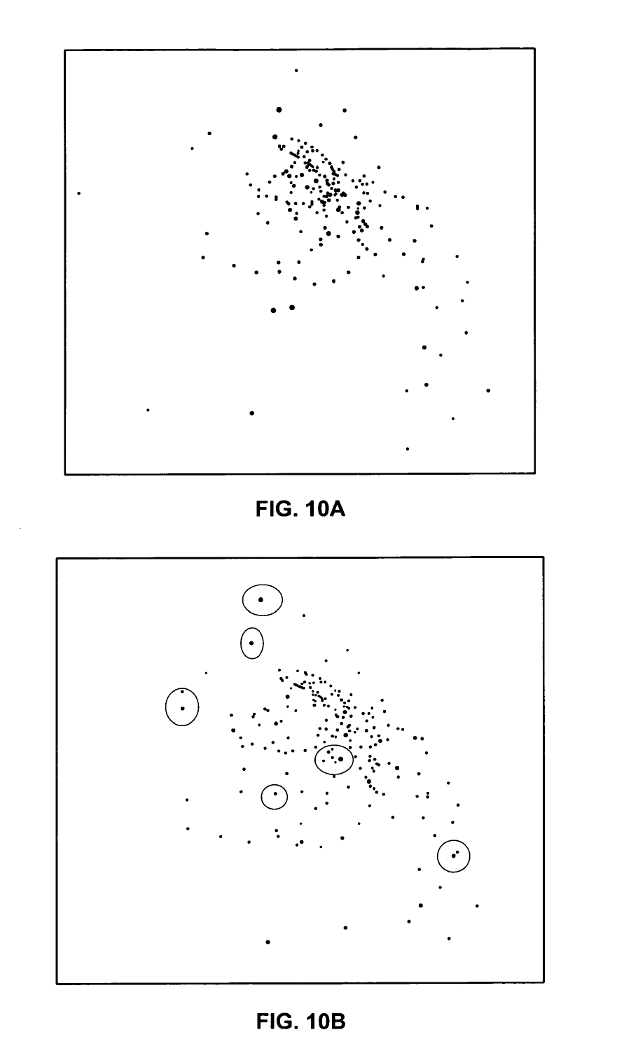

Each node of the base network is used to represent a protein or a gene. In this case, the two distinct molecular entities are referred to interchangeably, because a standard ID mapping table available from the UniProt database is used which can map between genes identified by standard gene symbols and corresponding proteins identified by unique UniProt identifiers. Each edge is used to represent an interaction relationship between two proteins. FIG. 10 shows AD gene terrain base network layouts. FIG. 10A shows the foundation layout of the data set before optimization, and FIG. 10B shows the foundation layout after optimization. After minimization, the most significant nodes are spread out and black circles indicate the regions of interests, which contain at least one highly significant AD protein.

Gene expression values are then used to render heights of the gene terrain visualizations. This rendering is based on the foundation layout and interpolation method described earlier. The height of each node is used to represent the gene expression value of each protein. The AD gene expression data used was collected from a published expression microarray data set, which derived from microarray analysis of the brain tissues from thirty-one individuals, which includes nine healthy individuals, seven incipient AD patients, eight moderate AD patients, and seven severe AD patients. The gene expression value for each gene is calculated from gene-mapped probe sets, each of which is indentified by its AFF_ID and contains a single gene expression value. Each probe set gene expression value was mapped to a gene expression value.

Algebraic averaging is used to compute the aggregated expression value if multiple probe set values can be mapped to a unique protein identified by its UNIPROT_ID. After this aggregation, 218 out of 625 protein nodes and 19 out of top 20 significant protein nodes remained.

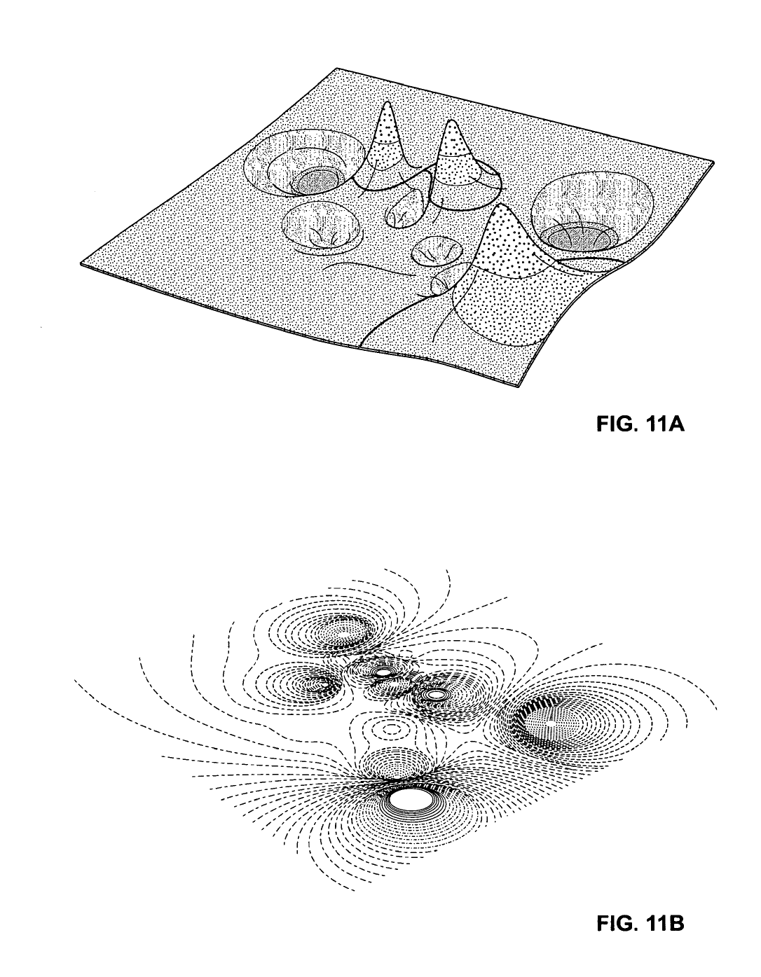

FIG. 11A shows an exemplary terrain surface and FIG. 11B shows an exemplary contour indicating gene expression data from the AD normal (control) group. Note that the height value in the z-direction is adjusted to a proper scale of gene expression suitable for display and exploration. The scale in the z-direction is different from the scale of grids used in the x-y plane.

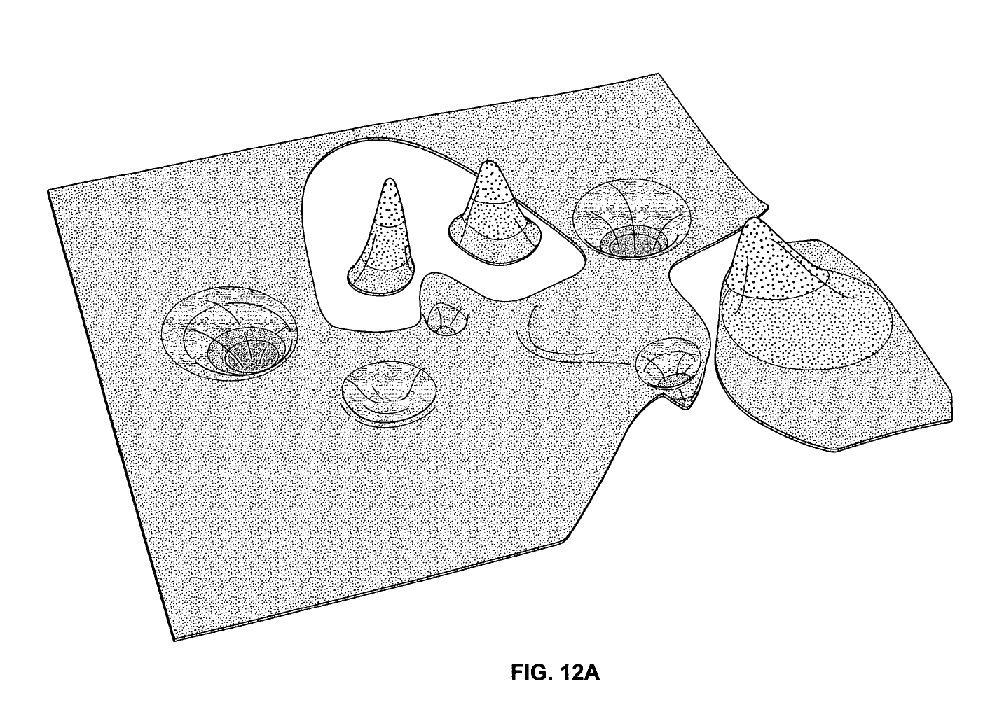

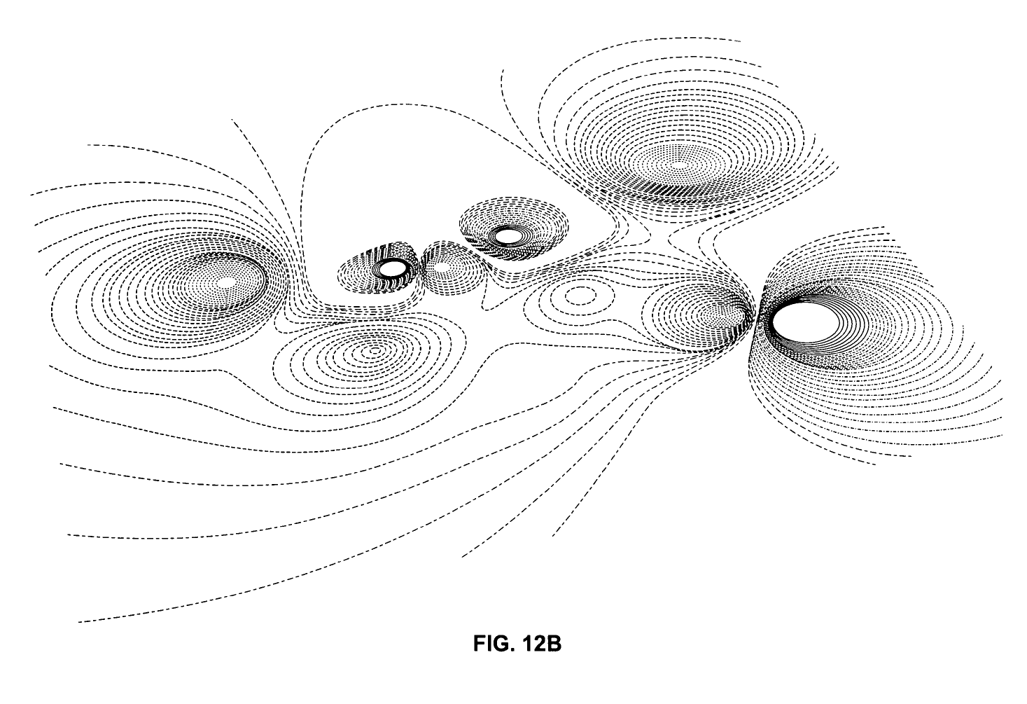

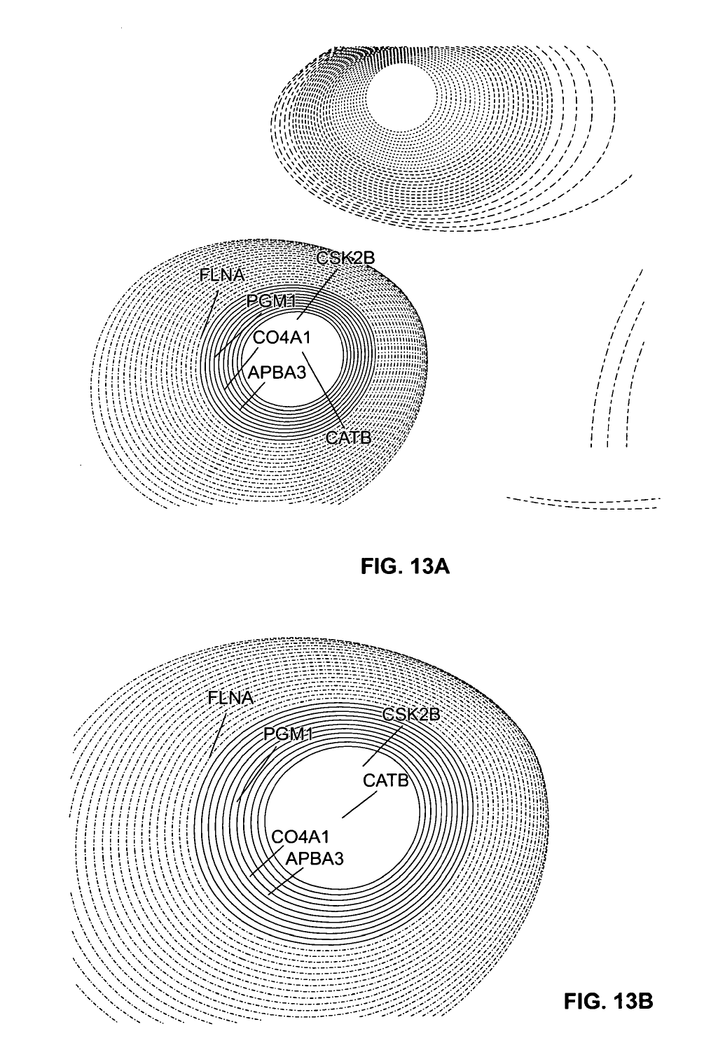

User interaction can be provided for visual exploration. The labels can be toggled on to support an overview of the distribution of protein nodes. The label of an individual protein can be toggled on by querying the name of the protein. To enable multi-scale visualization, a threshold T (T>0) can be set and only proteins whose height values are larger than T will be displayed. In this way, multiscale visualization can organize hundreds of proteins and gradually narrow down the search space by increasing the threshold value, T. Meanwhile, proteins can be grouped by different threshold and may yield biologically meaningful clusters. FIG. 12A shows the terrain with a protein threshold of T=3 and FIG. 12B is a contour visualization of this terrain. FIG. 13A shows a zoomed-in view of a portion of the contour of FIG. 12B, and FIG. 13B shows a further zoomed-in view of a portion of the contour of FIG. 12B. The zoom function can display details of local regions in the contour. The zoom function can also be done on a contour.

To support more advanced visual explorations, protein names in regions of interest can be shown by clicking the area. Note that only proteins whose heights are above the current threshold T and whose coordinates are within a circle centered at the clicking point with predefined radius a are shown. FIG. 13A shows all protein names in a peak area in the contour visualization. FIG. 13B is a further zoomed-in view to easier identify each protein's name.

To perform biomarker discoveries, the differential expression levels can be calculated as fold changes for each gene. An AD biomarker refers to a minimal set of consistently differentially expressed genes. To use AD visualization towards this purpose, the height of the terrains at each location of the gene can be represented with relative gene expression values from AD versus normal conditions instead of absolute gene expression values from normal samples. To do so, it was verified that the gene expression data sets obtained from the publication were already normalized. The absolute gene expression values were then averaged for all grouped individuals to their mean value. The AD patient groups (incipient, moderate, and severe) were then paired with the normal control group to derive relative gene expression. Relative or differential gene expressions are rendered as a new type of terrain sharing the same foundation layout of the terrain for absolute gene expressions. Relative gene expression values can be calculated according to standard gene expression analysis conventions as follows:

$$ReExp ( pro\_id ) = \{ \begin{matrix}\frac{Exp 2 ( pro\_id )}{Exp 1 ( pro\_id )} \cdot Exp 2 ( pro\_id ) \geq Exp 1 ( pro\_id ) \\ - \frac{Exp 1 ( pro\_id )}{Exp 2 ( pro\_id )} \cdot Exp 2 ( pro\_id ) < Exp 1 ( pro\_id ) ,\end{matrix}$$

where
ReExp(pro_id) represents the differential gene expression ratio for the diseased stage versus normal control condition for a given protein with pro_id as the identifier,
Exp1(pro_id) is the absolute gene expression value for the same protein under condition 1, and
Exp2(pro_id) is the absolute gene expression value for the same protein under condition 2.

Therefore, differential gene expression values have an absolute value greater than or equal to 1. To filter differential gene expression values due to natural variability of gene expressions, only changes beyond 5% of normal controls were considered, or ≧1.05 and <−1.05 cases, when considering candidate biomarkers for inclusion in the lymphoma related biomarker panel.

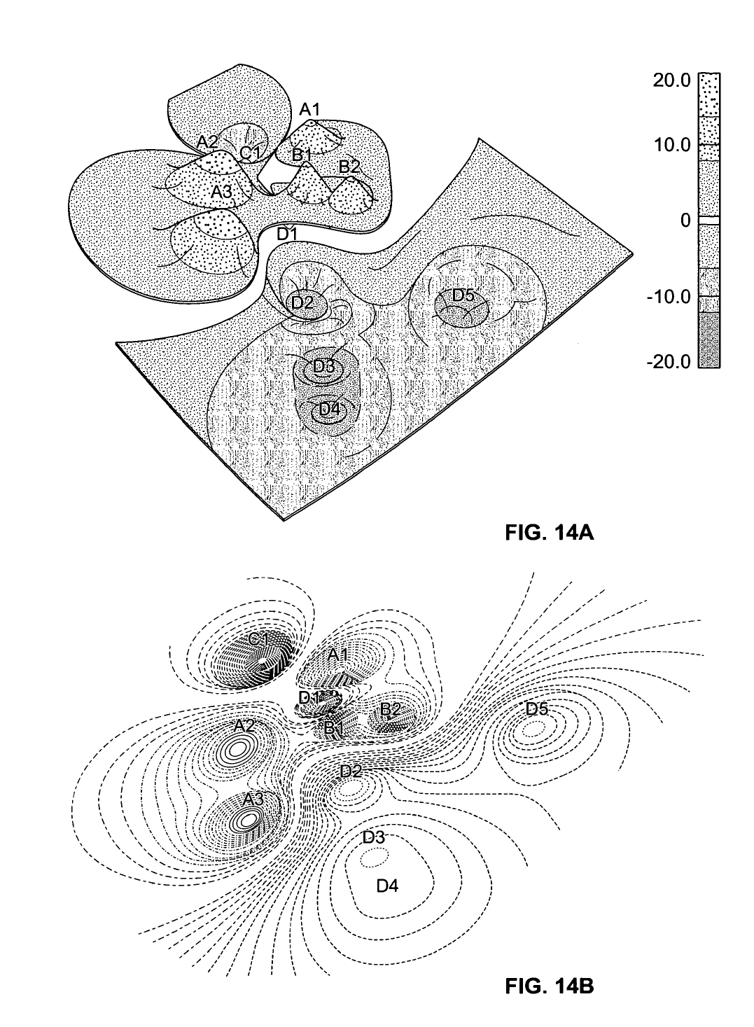

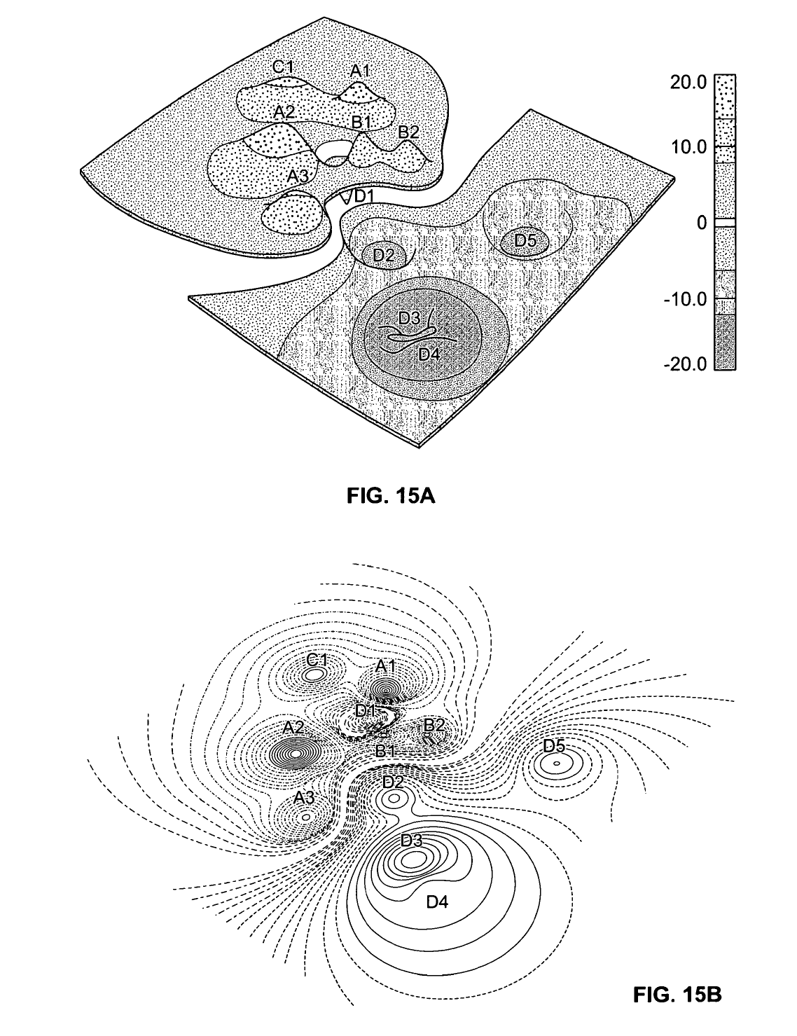

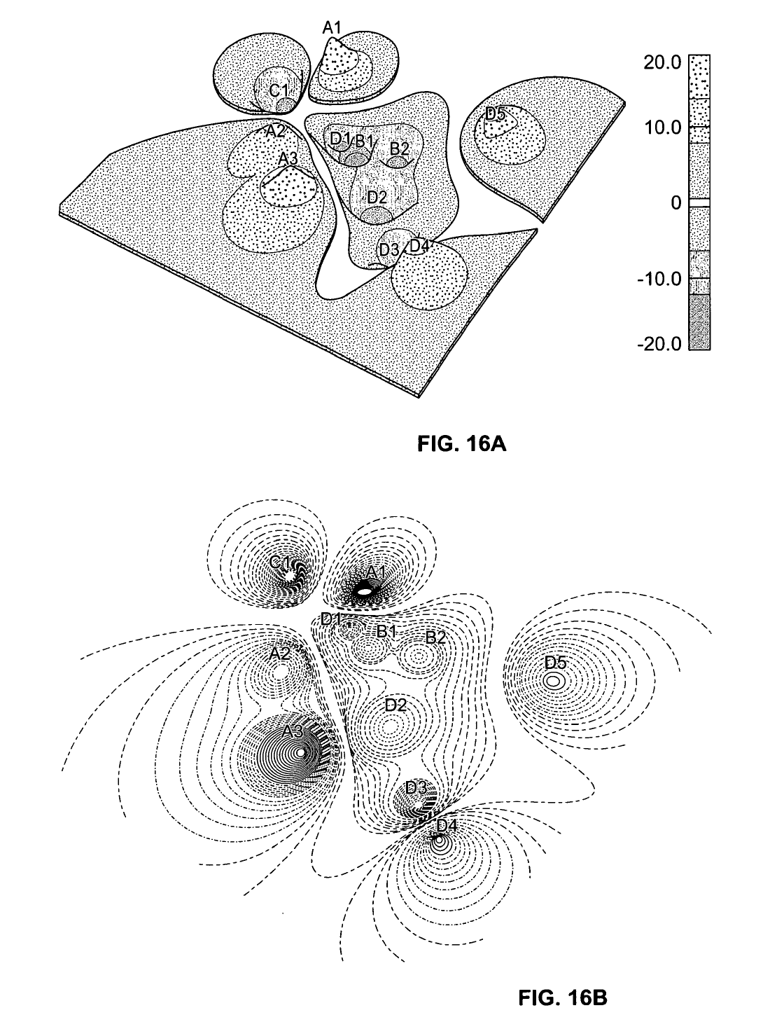

FIGS. 14-16 show a series of differential expression surfaces and contours. FIG. 14A shows a differential expression terrain surface for control versus incipient condition, and FIG. 14B shows a differential expression contour for control versus incipient condition. FIG. 15A shows a differential expression terrain surface for control versus moderate condition, and FIG. 15B shows a differential expression contour for control versus moderate condition. FIG. 16A shows a differential expression terrain surface for control versus severe condition, and FIG. 16B shows a differential expression contour for control versus severe condition. A threshold of height values was set for the surface. The portion of the surface with height values out of the range is set to be transparent in the terrain, and no contour is displayed for these portions. Peak and valley areas are colored separately. Red can be used to represent an over-expressed value, but here we use red to represent areas with comparatively lower height value as the surfaces are control versus condition.

From FIGS. 14-16, peaks and valleys can be observed in the terrain surface maps and rings of concentric circles in the contour maps. These distinct visual features serve as ‘visual cues’, allowing a researcher to quickly comprehend the results of AD differential gene expressions in their biological context. In the terrain images, peaks are clearly identifiable with colors ranging from red, yellow, green, to blue. The major peaks and valleys are labeled for easy comparisons between different panels in FIGS. 14-16. The area with height value within a certain range is set to transparency to separate features. With these visual representations, several observations can be readily made.
(1) Peaks A1, A2 and A3 are present in all panels, indicating that relative to controls, the AD conditions lack the expressions for these genes. The proteins in these peak areas, especially those determined to have significant links to AD (protein nodes with high weight scores from previous studies), are candidate AD diagnostic biomarkers. Similarly, valleys D1 and D2 can also be diagnostic biomarkers.
(2) The height of peak A1 increases as AD progressed in stages. Therefore, proteins in this peak can be considered candidate prognostic biomarkers.
(3) Peaks B1 and B2 disappear in the severe form of AD, and valley D3 appears in the severe form of AD. This makes the up-regulation of proteins within peaks B1 and B2 as well as down-regulation of proteins within peaks D3 candidate staging biomarkers.
(4) The small peak C1 appears in moderate AD versus control normal whereas it is transformed to a valley in incipient or severe differential AD gene expression profiles. The inconsistent behavior of the protein in the area of C1 poses an interesting question.

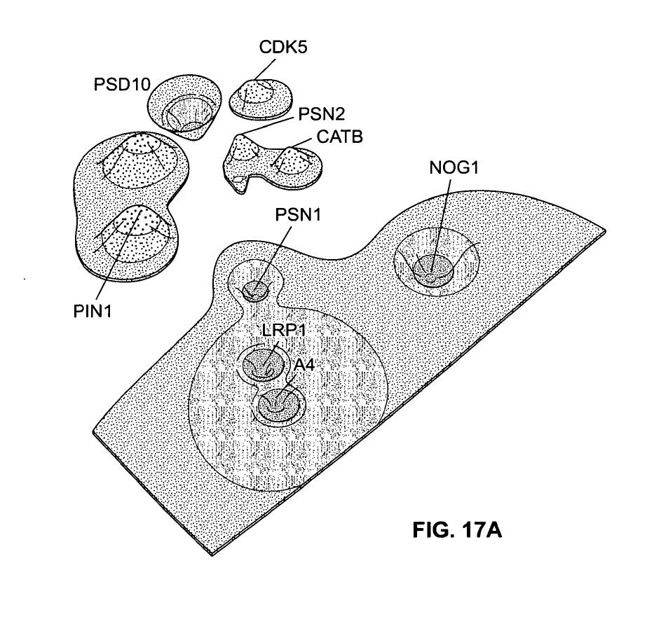

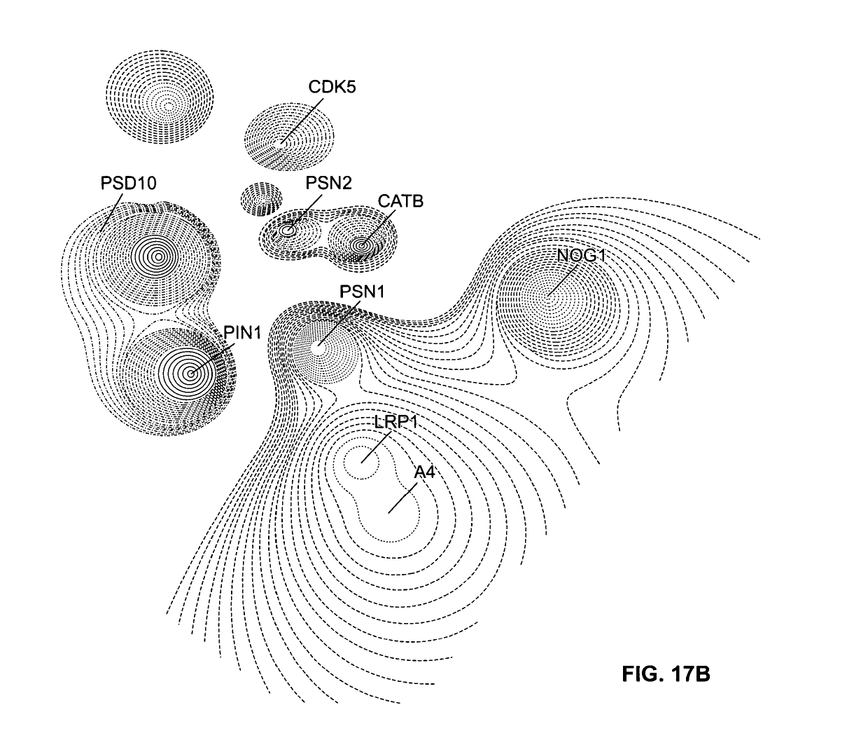

We further identified proteins of interest within the peaks/valleys of the terrain and contours. This can be performed by clicking on a region of interest and toggling on gene labels. FIG. 17A shows the results of such interactive visual querying, in which the name of proteins in the peak or valleys with differential gene expression levels above thresholds in control versus incipient AD is shown. FIG. 17B displays the contour map corresponding to FIG. 17A. Using the interactive functionality introduced above, more protein names will appear in the region of interest by decreasing the threshold value.

By examining all relative terrains, the prognostic biomarker in peak A1 was identified to be mainly explained by protein ‘CDK5_HUMAN’ in the top 20 significant proteins shown in FIG. 9. The link between CDK5 and AD has been well supported by prior biomedical studies but the role of CDK5 as a potential AD biomarker has not been previously reported.

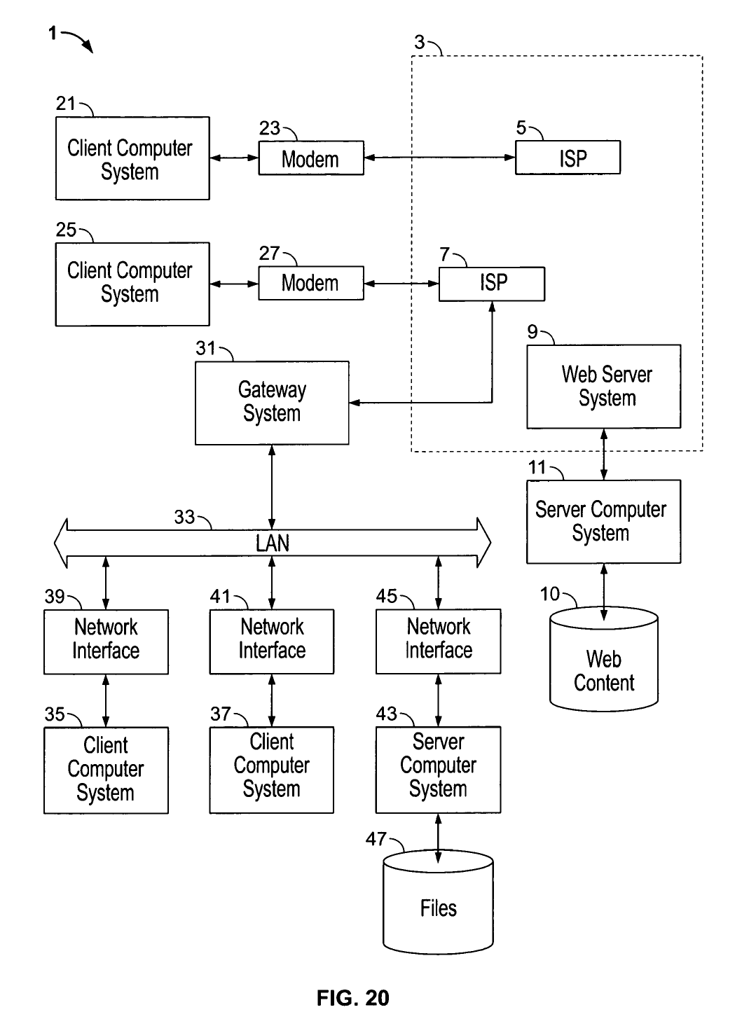

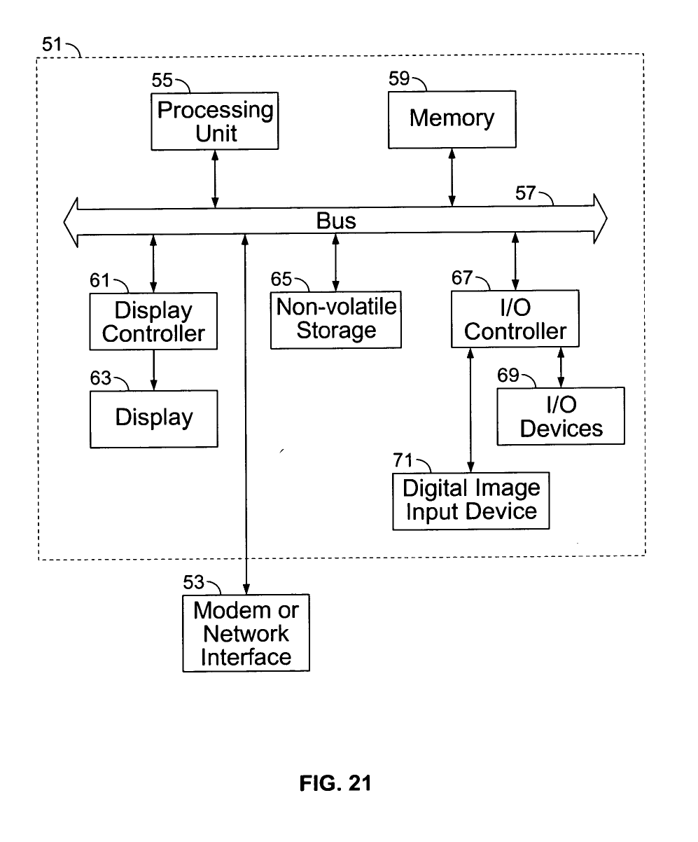

The following description of FIGS. 20 and 21 are intended to provide an overview of exemplary computer hardware and other operating components suitable for performing the methods of the invention described above. However, it is not intended to limit the applicable environments. One of skill in the art will immediately appreciate that the invention can be practiced with other computer system configurations, including hand-held devices, multiprocessor systems, microprocessor-based or programmable consumer electronics, network PCs, minicomputers, mainframe computers, and the like. The invention can also be practiced in distributed computing environments where tasks are performed by remote processing devices that are linked through a communications network, such as a local area network (LAN), wide-are network (WAN), or over the Internet.

FIG. 21 shows several computer systems 1 that are coupled together through a network 3, such as the Internet. The term “Internet” as used herein refers to a network of networks which uses certain protocols, such as the TCP/IP protocol, and possibly other protocols such as the hypertext transfer protocol (HTTP) for hypertext markup language (HTML) documents that make up the World Wide Web (web). The physical connections of the Internet and the protocols and communication procedures of the Internet and other networks are well known to those of skill in the art. Access to the Internet 3 is typically provided by Internet service providers (ISP), such as the ISPs 5 and 7. Users on client systems, such as client computer systems 21, 25, 35, and 37 obtain access to the Internet through the Internet service providers, such as ISPs 5 and 7. Access to the Internet allows users of the client computer systems to access databases, exchange information, receive and send messages, and view documents, such as documents which have been prepared in the HTML format. These documents are often provided by web servers, such as web server 9 which is considered to be “on” the Internet. Often these web servers are provided by the ISPs, such as ISP 5, although a computer system can be set up and connected to the Internet without that system being also an ISP as is well known in the art.

The web server 9 is typically at least one computer system which operates as a server computer system and is configured to operate with the protocols of the World Wide Web and is coupled to the Internet. Optionally, the web server 9 can be part of an ISP which provides access to the Internet for client systems. The web server 9 is shown coupled to the server computer system 11 which itself is coupled to web content database 10, which can be considered a form of a media or information database. It will be appreciated that while two computer systems 9 and 11 are shown in FIG. 20, the web server system 9 and the server computer system 11 can be one computer system having different software components providing the web server functionality and the server functionality provided by the server computer system 11.

Client computer systems 21, 25, 35, and 37 can each, with the appropriate software, view HTML pages provided by the web server 9. The ISP 5 provides Internet connectivity to the client computer system 21 through the modem interface 23 which can be considered part of the client computer system 21. The client computer system can be a personal computer system, a network computer, a Web TV system, a handheld device, or other such computer system. Similarly, the ISP 7 provides Internet connectivity for client systems 25, 35, and 37, although as shown in FIG. 20, the connections are not the same for these three computer systems. Client computer system 25 is coupled through a modem interface 27 while client computer systems 35 and 37 are part of a LAN. While FIG. 20 shows the interfaces 23 and 27 as generically as a “modem,” it will be appreciated that each of these interfaces can be an analog modem, ISDN modem, cable modem, satellite transmission interface, or other interfaces for coupling a computer system to other computer systems. Client computer systems 35 and 37 are coupled to a LAN 33 through network interfaces 39 and 41, which can be Ethernet network or other network interfaces. The LAN 33 is also coupled to a gateway computer system 31 which can provide firewall and other Internet related services for the local area network. This gateway computer system 31 is coupled to the ISP 7 to provide Internet connectivity to the client computer systems 35 and 37. The gateway computer system 31 can be a conventional server computer system. Also, the web server system 9 can be a conventional server computer system.

Alternatively, as well-known, a server computer system 43 can be directly coupled to the LAN 33 through a network interface 45 to provide files 47 and other services to the clients 35, 37, without the need to connect to the Internet through the gateway system 31.

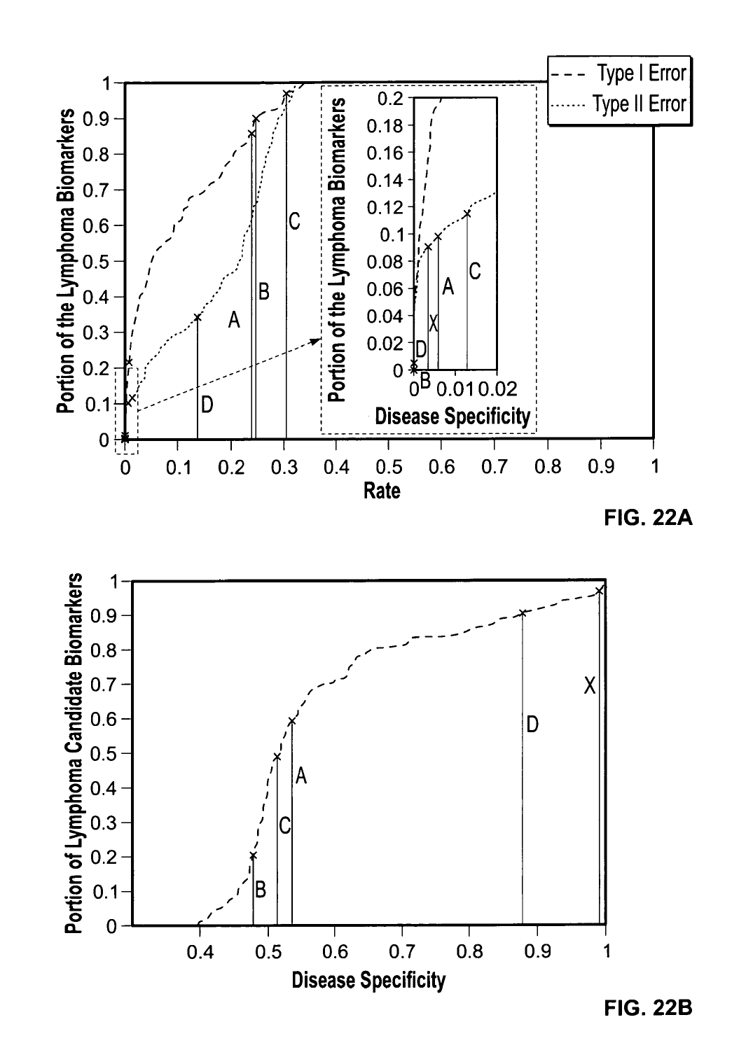

FIG. 22 shows an exemplary computer system that can be used as a client computer system or a server computer system or as a web server system. It will also be appreciated that such a computer system can be used to perform many of the functions of an Internet service provider, such as ISP 5. The computer system 51 interfaces to external systems through the modem or network interface 53. It will be appreciated that the modem or network interface 53 can be considered to be part of the computer system 51. This interface 53 can be an analog modem, ISDN modem, cable modem, token ring interface, satellite transmission interface, or other interfaces for coupling a computer system to other computer systems. The computer system 51 includes a processing unit 55, which can be a conventional microprocessor such as microprocessors made by Intel or AMD. Memory 59 is coupled to the processor 55 by a bus 57. Memory 59 can be dynamic random access memory (DRAM), static RAM (SRAM) or other types of memory. The bus 57 couples the processor 55 to the memory 59 and also to non-volatile storage 65 and to display controller 61 and to the input/output (I/O) controller 67. The display controller 61 controls a display on a display device 63 which can be a cathode ray tube (CRT), liquid crystal display (LCD) or other type of display device. The input/output devices 69 can include a keyboard, disk drives, printers, a scanner, and other input and output devices, including a mouse or other pointing device. The display controller 61 and the I/O controller 67 can be implemented with conventional well known technology. A digital image input device 71 can be a digital camera which is coupled to an I/O controller 67 in order to allow images from the digital camera to be input into the computer system 51. The non-volatile storage 65, an example of a “computer-readable storage medium” and a “machine-readable storage medium”, is often a magnetic hard disk, an optical disk, or another form of storage for large amounts of data. Some of this data is often written, by a direct memory access process, into memory 59 during execution of software in the computer system 51. One of skill in the art will immediately recognize that the terms “computer-readable medium” and “machine-readable medium” include any type of “computer-readable storage medium” and “machine-readable storage medium” (e.g., storage device) that is accessible by the processor 55.

It will be appreciated that the computer system 51 is one example of many possible computer systems which have different architectures. For example, personal computers based on an Intel microprocessor often have multiple buses, one of which can be an input/output (I/O) bus for the peripherals and one that directly connects the processor 55 and the memory 59 (often referred to as a memory bus). The buses are connected together through bridge components that perform any necessary translation due to differing bus protocols.

It will also be appreciated that the computer system 51 is controlled by operating system software which includes a file management system, such as a disk operating system, which is part of the operating system software. One example of operating system software with its associated file management system software is the Windows family of operating systems from Microsoft Corporation of Redmond, Wash., and their associated file management systems. The file management system is typically stored in the non-volatile storage 65 and causes the processor 55 to execute the various acts required by the operating system to input and output data and to store data in memory, including storing files on the non-volatile storage 65.

The following examples are offered by way of illustration and not limitation.

### EXPERIMENTAL

### Example 1

### Biomarker Panel Development

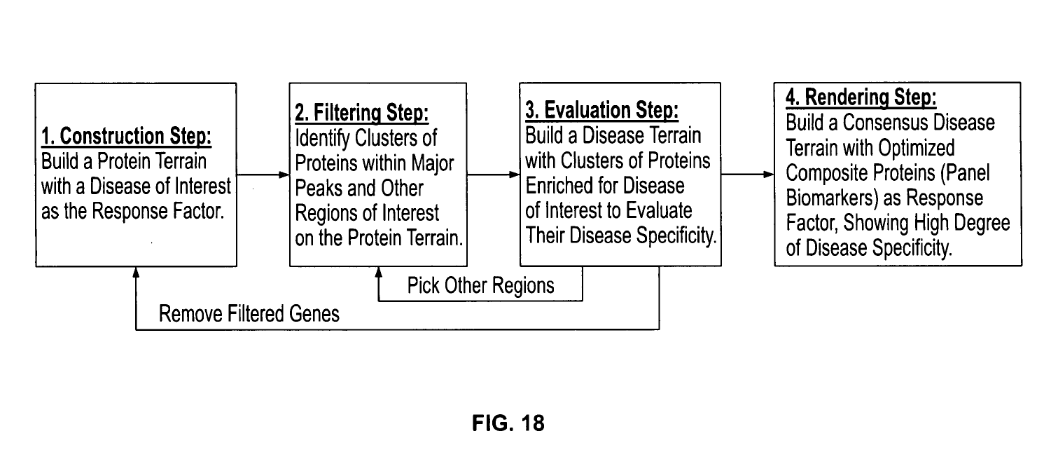

The lack of specific single biomarker for many disease biomarker applications is a challenge for biomarker development today. An approach shown in FIG. 18 was used to iteratively design a biomarker panel. The approach includes four steps: a construction step where a protein terrain is built with a disease of interest as the response factor; a filtering step where clusters of proteins within major peaks and other regions of interest are identified on the protein terrain; an evaluation step where a disease terrain is built with clusters of proteins enriched for the disease of interest to evaluate their disease specificity; and a rendering step where a consensus disease terrain is built with optimized composite proteins (panel biomarkers) as response factors showing a high degree of specificity. This can be an iterative process where other regions on the protein terrain can be selected and filtered genes can be removed.

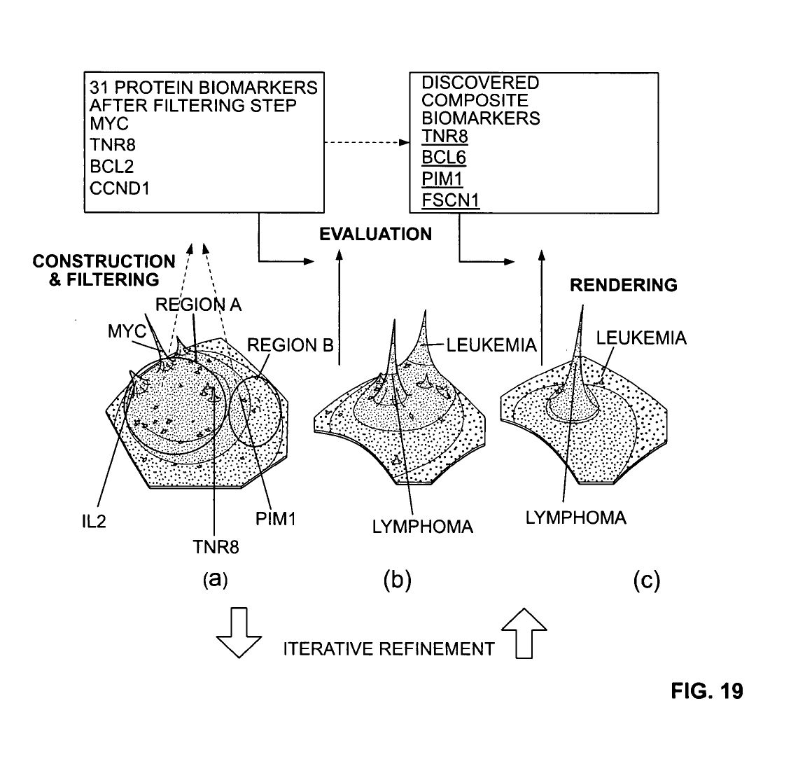

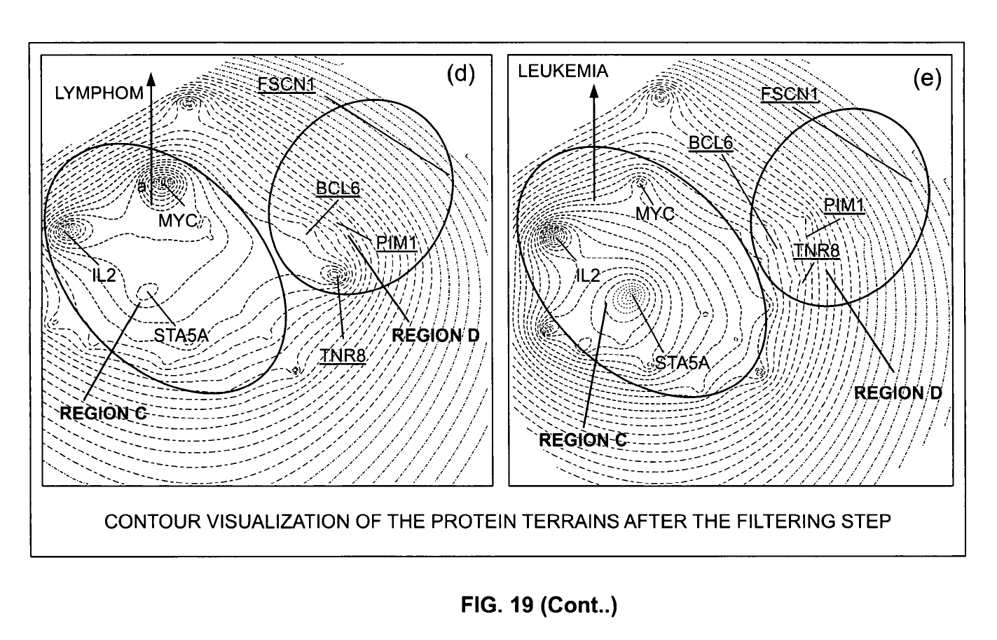

Lymphoma was used as a case study, since several subtypes of late-stage lymphoma are known to be clinically co-occurring with leukemia and our visual analytic analysis of several known single protein markers for lymphoma on disease terrain confirmed their non-specific performance between lymphoma and leukemia. Both TNFRSF8 and BCL6 have been found to have strong cell-based differential expression patterns between normal and non-Hodgkin's lymphoma cell lines or tissue samples. PIM-1, whose cell expression is broadly spread in many types of cancers, has recently been reported to be a good drug treatment prognosis biomarker in mantle cell lymphoma. Similarly, soluble FSCN1 receptor (TNF Type I receptor) has long been reported to be reversely associated with lymphoma prognosis. The results of this correlative visual analysis are shown in FIG. 19.

Following the work flow outlined in FIG. 18 for lymphoma panel biomarker development, the results are shown beginning with the initial construction step where a lymphoma protein terrain was built by choosing the HAPPI-3 base network (see FIG. 19(a)). Among all the candidate cancer biomarkers used for this study, 169 curated lymphoma candidate biomarkers are covered.

In the filtering step, regions A and B (labeled in FIG. 19(a)) were identified as regions of interest on the protein terrain. Region A contains major clustered peaks characteristic of the entire lymphoma protein terrain. Region B is a peripheral area of Region A with extended surface slopes and small “buds.” Together regions A and B contain 31 of 169 curated lymphoma candidate biomarkers. In this study, the candidate biomarkers within these two regions were focused on and used to build the initial panel (shown in the table preceding FIG. 19(b)).

In the evaluation step, the lymphoma disease specificity was evaluated of an identified cluster of candidate biomarkers from the filtering step. The difference here compared to evaluating a single protein biomarker is that a consensus disease terrain is rendered for all filtered proteins in a panel. In the consensus disease terrain shown in FIG. 19(b), the same base disease association network (type CNL II) was used but used a simple average of all the association strengths of genes in the panel between each region on the disease base network and lymphoma as the interpolated response factor. This consensus disease terrain contains two dominating peaks, one for lymphoma and the other for leukemia.

Before rendering the final disease terrain, it is usually necessary to go back to earlier steps to remove filtered genes and pick other regions of interest iteratively, using consensus disease terrain visualization with the panel of revised set of proteins as the response factor. Contours of the two protein terrains are shown, one for lymphoma (FIG. 19(d)) and the other for leukemia (FIG. 19(e)), during iterative refinements. In both contours, a common peak region, Region C, and an outside slope region, Region D, are identified. Twenty out of thirty-one curated candidate proteins are located in Region C, and these were filtered out for concerns that these proteins would not be distinguishable between the two cancer types being inside the common peak region. In Region D, proteins TNR8_HUMAN (Lymphocyte activation antigen, TNFRSF8) and BCL6_HUMAN (B-Cell Lymphoma 6, BCL6) were kept because they show peaks only in the lymphoma protein terrain contour but not in the leukemia protein terrain contour. Additional evaluations of what other proteins to keep in Region D were performed, and the candidates were evaluated for specificity. The involvement of genes and proteins in multiple disease pathways makes the existence of genes and proteins specifically linked to a disorder uncertain. Two more proteins, PIM1_HUMAN (Proto-oncogene serine/threonine-protein kinase, PIM-1) and FSCN1_HUMAN (Fascin, p55), were thus iteratively found and added to the biomarker panel for lymphoma.

In the rendering step, a consensus disease terrain was built for the completed panel of four biomarkers (see FIG. 19(c)). A comparison of FIG. 19(b) (before refinement) and 16(c) (after refinement) shows dramatically improved lymphoma disease specificity. This new multi-panel biomarker consists of a manageable number of proteins, with both high sensitivity (high peak) and high specificity (unique peak).

### Example 2

### Validation of a Lymphoma Related Biomarker Panel

A four member lymphoma related biomarker panel comprising TNFRSF8, FSCN1, BCL6 and PIM1 and candidate biomarkers were assessed for sensitivity and specificity in a prospective manner. The performance of a newly found biomarker panel can be validated by measuring their disease sensitivity and disease specificity. For this exemplary experiment, the disease sensitivity is defined by the results of bi-classification on microarray expression samples, where the case is lymphoma samples and the control is normal samples. For this exemplary experiment, the disease specificity is defined by the results of bi-classification on microarray expression samples where the case is leukemia samples and the control is lymphoma samples.

Microarray results derived from 25 normal blood samples, 29 lymphoblastoid lymphoma cell line tissue samples and 34 B-cell chronic lymphocytic leukemia cell lines were obtained from a functional genomics study by the National Center for Biotechnology Information (NCBI) http://www.ncbi.nlm.nih.gov/projects/geo/query/acc.cgi?acc=GSE2350. The data was preprocessed and normalized as described elsewhere herein. 156 out of the 169 candidate lymphoma biomarker genes are found in the GeneChip probe sets used in the NCBI functional genomics study.

The disease sensitivity was characterized for two types of errors: Type I error is the ratio between lymphoma samples (in this study, lymphoblastoid lymphoma cell line tissue samples) classified as normal and the total number of lymphoma samples; Type II error is the ratio between the number of normal samples misclassified as lymphoma and the total number of normal samples. A preferred Type I error rate for a lymphoma related biomarker panel is less than 20%, 19%, 18%, 17%, 16%, 15%, 14%, 13%, 12%, 11%, 10%, 9%, or 8%; a more preferred Type I error rate for a lymphoma related biomarker panel is less than 7%, 6%, 5%, or 4%; a yet more preferred Type I error rate for a lymphoma related biomarker is less than 3%, 2%, 1%, 0.9%, 0.8%. 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01%. A preferred Type II error rate for a lymphoma related biomarker panel is less than 20%, 19%, 18%, 17%, 16%, 15%, 14%, 13%, 12%, 11%, 10%, 9%, or 8%; a more preferred Type II error rate for a lymphoma related biomarker panel is less than 7%, 6%, 5%, or 4%; a yet more preferred Type II error rate for a lymphoma related biomarker is less than 3%, 2%, 1%, 0.9%, 0.8%. 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01%. It is recognized that assessing a Type I or Type II error rate involves a statistically significant population size for both normal and lymphoma exhibiting subjects. The disease specificity is defined as the ratio between the lymphoma samples in the lymphoma-dominated class and the total number of samples in that class when comparing leukemia samples and lymphoma samples. Results of disease specificity and disease analysis are presented in FIG. 22.

Cumulative density function (CDF) analysis was performed on the four member lymphoma related biomarker panel, TNFRSF8, FSCN1, BCL6 and PIM1, and the other 152 candidate lymphoma biomarkers. In CDF, the x-value is the performance, e.g. Type I error, and the y-value is the portion of bench marks whose performance is less than x. In the case of Type I and II errors, the lower the y-value of the detected panel indicates the more accurately the panel classified the normal and lymphoma samples. Results from the analysis are presented in FIG. 22, panel A.

Specificity of the four member lymphoma related biomarker panel, TNFRSF8, FSCN1, BCL6 and PIM1, and the other 152 candidate lymphoma biomarkers were analyzed for the percentage of all lymphoma samples in the lymphoma dominated class when comparing leukemia samples against lymphoma samples. In the case of disease specificity, the higher the y value of the detected panel indicates the better the panel distinguishes lymphoma conditions from leukemia conditions. Results from the analysis are presented in FIG. 22, panel B.

### Example 3

### Normalization and Pre-processing of Microarray Expression Data

Microarray results derived from 25 normal blood samples, 29 lymphoblastoid lymphoma cell line tissue samples and 34 B-cell chronic lymphocytic leukemia cell lines were obtained from a functional genomics study by the National Center for Biotechnology Information (NCBI) http://www.ncbi.nlm.nih.gov/projects/geo/guery/acc.cgi?acc=GSE2350. All of the eighty-eight samples were aligned. The microarray results from each sample each had 12533 probes. The data were normalized by the expression level of identified “house keeping” probes. Two steps were used in performing “house keeping” probe normalization.

The first step of “house keeping” probe normalization was a quantile normalization check. The data set was checked to see if it needed any routine normalization, e.g. quantile normalization. For each of the 88 samples, the top 5% percentile and bottom 5% percentile expressed probes were excluded, and the mean and standard deviation of the expression for the remaining probes were calculated. The standard deviation from all samples in this study was 8.22. The normalization checks were repeated by temporarily removing the top and bottom 10% percentile, and then removing the top and bottom 25% percentile. In each check the standard deviations among the mean values was acceptably small, indicating this data set was quantile normalized.

The second step of “house keeping” probe normalization was normalization based on the “house keeping” probes. The “house-keeping” probes were first identified. “House-keeping” probes are distinguished from probes that barely function, because “house-keeping” probes have relatively more stable expression across all the samples, while the expressions of barely functioning probes are low and not reliable due to the unavoidable artifacts introduced by the chips. To identify the house-keeping probes, the P/M/A calls were examined for probe expressions in all samples used, and the maximum expression marked with absence call (41.4 in this study) was used as the minimal threshold, T, for presence and absence. Probes that have intensity values dropping below the threshold were temporarily removed in minimal 5% of all the samples used (5% is to assume that some samples may be outliers). In this study, 4912 out of 12533 probes remained. For the remaining probes, the bottom 100 probes with the least variance across all samples were identified as the “house-keeping” probes. The average of the expressions of “house-keeping” probes was denoted as IO (in this study, IO was 98.23), the base line.

The baseline from “house keeping” probes was then used as the “internal standard” to normalize each expression. Each new expression value was normalized with regard to the standard, i.e. the normalized expression IX′ is computed as max (0, (IX−T)/(IO−T)). Note that this calculation sets expressions lower than the base line to zero.

The probes expressions were correlated with genes, and then those genes were mapped to obtain the expression of the 169 candidate lymphoma biomarkers. The 169 candidate lymphoma biomarkers were from 762 candidate biomarkers which derived from the HAPPI database, and used for the construction of the candidate biomarker protein interaction network. When multiple gene symbols mapped to the same protein Uniprot ID, a simple linear average was used to calculate the expression of the protein. As a result, 156 out of the 169 candidate lymphoma biomarkers were found in the GeneChip Probe set. Among them, are four lymphoma related biomarkers in the newly detected panel, i.e. TNFRSF8, FSCN1, PIM1 and BCL6.

Expression of the four lymphoma related biomarkers was used as a four dimension feature vector for each sample, for classification. The remaining 156 single candidate lymphoma biomarkers were used as comparisons. Hierarchical clustering is then used to cluster the feature vectors of samples, in order to approximate the best possible bi-class classification results. In the hierarchical clustering, the “Euclidean” default distance measure, and “mean” default linkage method is used. The results are compared to the known annotations, and the errors define the two performance criteria: disease sensitivity and specificity.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

Having described the invention with reference to the exemplary embodiments, it is to be understood that it is not intended that any limitations or elements describing the exemplary embodiment set forth herein are to be incorporated into the meanings of the patent claims unless such limitations or elements are explicitly listed in the claims. Likewise, it is to be understood that it is not necessary to meet any or all of the identified advantages or objects of the invention disclose herein in order to fall within the scope of any claims, since the invention is defined by the claims and since inherent and/or unforeseen advantages of the present invention may exist even though they may not be explicitly discussed herein.

Although the foregoing invention has been described in some detail by way of illustration and example for purposes of clarity of understanding, it will be obvious that certain changes and modifications may be practiced within the scope of the appended claims.

That which is claimed:

1. A kit for evaluating expression of at least three biomarkers from a lymphoma related biomarker panel wherein said lymphoma related biomarker panel comprises TNFRSF8, FSCN1, BCL6 and PIM1; said kit comprising:
(a) a first biomarker detection reagent capable of preferentially detecting expression of a first biomarker selected from said lymphoma related biomarker panel;
(b) a second biomarker detection reagent capable of preferentially detecting expression of a second biomarker selected from said lymphoma related biomarker panel; and
(c) a third biomarker detection reagent capable of preferentially detecting expression of a third biomarker selected from said lymphoma related biomarker panel.

2. The kit of claim 1 further comprising a fourth biomarker detection reagent capable of preferentially detecting expression of a fourth biomarker selected from said lymphoma related biomarker panel.

3. The kit of claim 2, wherein said first biomarker detection regeant preferentially detects expression of TNFRSF8, said second biomarker detection reagent preferentially detects expression of FSCN1, said third biomarker detection reagent preferentially detects expression of BCL6, and said fourth biomarker detection reagent preferentially detects expression of PIM1.

4. A kit for characterizing a lymphoma related disorder in a subject at risk for a lymphoma related disorder comprising at least three biomarker detection reagents for at least three biomarkers from a lymphoma related biomarker panel wherein said lymphoma related biomarker panel comprises TNFRSF8, FSCN1, BCL6 and PIM1; said kit comprising:
(a) a first biomarker detection reagent capable of preferentially detecting expression of a first biomarker selected from said lymphoma related biomarker panel;
(b) a second biomarker detection reagent capable of preferentially detecting expression of a second biomarker selected from said lymphoma related biomarker panel; and
(c) a third biomarker detection reagent capable of preferentially detecting expression of a third biomarker selected from said lymphoma related biomarker panel.

5. A method of characterizing a lymphoma related disorder in a subject at risk for a lymphoma related disorder comprising the steps of:
(a) providing a biological sample obtained from said subject;
(b) evaluating expression in said sample of at least three biomarkers from a lymphoma related biomarker panel, wherein said lymphoma related biomarker panel comprises TNFRSF8, FSCN1, BCL6 and PIM1;
(c) comparing said expression of said biomarkers in said sample with a predetermined standard;
(d) identifying said expression of said biomarkers as altered or unaltered; and
(e) characterizing said lymphoma related disorder as lymphoma when said expression of said at least three biomarkers is altered.

6. The method of claim 5, comprising evaluating expression in said sample of at least four biomarkers from said lymphoma related biomarker panel.

7. The method of claim 5, wherein at least three of said biomarkers are selected from the group consisting of TNFRSF8, FSCN1, BCL6 and PIM1.

8. The method of claim 5 wherein said subject is a mammal.

9. The method of claim 8 wherein said mammal is selected from the group comprising humans, bovines, equines, murines, ovines, caprines, lapines, canines and swine.

10. The method of claim 5 wherein the Type I error rate is less than 20%.

11. The method of claim 5 wherein the Type II error rate is less than 20%.

12. The method of claim 5 wherein said altered expression of each said biomarker differs from said predetermined standard by at least 0.001%.

13. The method of claim 5 wherein said altered expression is decreased expression or increased expression.

14. A method of optimizing therapeutic efficacy associated with treatment of a lymphoma related disorder in a subject at risk for a lymphoma related disorder comprising the steps of:
(a) providing a biological sample obtained from said subject;
(b) evaluating expression in said sample of at least three biomarkers from a lymphoma related biomarker panel, wherein said lymphoma related biomarker panel comprises TNFRSF8, FSCN1, BCL6 and PIM1;
(c) comparing expression of said biomarkers with a predetermined standard;
(d) identifying said expression of said biomarkers as altered or unaltered; and
(e) administering a lymphoma preferred course of treatment to said subject when said expression of said at least three biomarkers in said panel is altered.

15. The method of claim 14, comprising evaluating expression in said sample of at least four biomarkers from said lymphoma related biomarker panel.

16. The method of claim 14, wherein at least three of said biomarkers are selected from the group consisting of TNFRSF8, FSCN1, BCL6 and PIM1.

17. A method of optimizing therapeutic efficacy associated with treatment of a lymphoma related disorder in a subject at risk for a lymphoma related disorder comprising the steps of:
(a) providing a biological sample obtained from said subject;
(b) evaluating expression in said sample of at least three biomarkers from a lymphoma related biomarker panel, wherein said lymphoma related biomarker panel comprises TNFRSF8, FSCN1, BCL6 and PIM1;
(c) comparing expression of said biomarkers with a predetermined standard;
(d) identifying said expression of said biomarkers as altered or unaltered; and
(e) administering a leukemia preferred course of treatment to said subject when said expression of said at least three biomarkers in said panel is unaltered.

18. The method of claim 17, comprising evaluating expression in said sample of at least four biomarkers from said lymphoma related biomarker panel.

19. The method of claim 17, wherein at least three of said biomarkers are selected from the group consisting of TNFRSF8, FSCN1, BCL6 and PIM1.

20. A method of identifying a subject at risk for a lymphoma related disorder comprising the steps of:
(a) providing a biological sample obtained from said subject;
(b) evaluating expression of at least three biomarkers from a lymphoma related biomarker panel comprising TNFRSF8, FSCN1, BCL6 and PIM1;
(c) comparing expression of said biomarkers in said lymphoma related biomarker panel with a predetermined standard;
(d) identifying said expression of said biomarkers as altered or unaltered; and
(e) identifying said subject as being at risk for lymphoma when said expression of said at least three biomarkers in said biomarker panel is altered.

21. A method of identifying a subject at risk for a lymphoma related disorder comprising the steps of:
(a) providing a biological sample obtained from said subject;
(b) evaluating expression of at least three biomarkers from a lymphoma related biomarker panel comprising TNFRSF8, FSCN1, BCL6 and PIM1;
(c) comparing expression of said biomarkers in said lymphoma related biomarker panel with a predetermined standard;
(d) identifying said expression of said biomarkers as altered or unaltered; and
(e) identifying said subject as being at risk for leukemia when said expression of said at least three biomarkers in said biomarker panel is unaltered.

22. A visualization method for determination of candidate biomarker panels for a disease of interest, the method comprising:
accessing a protein database containing data regarding genes and protein;
accessing a disease database containing data regarding diseases;
constructing a protein base network and protein terrain using the data from the protein database for a disease of interest; the constructing being done with a computer processor;
displaying the protein terrain on a computer display device;
constructing a disease base network and disease terrain using the data from the disease database for the proteins of the protein base network, the constructing being done with a computer processor;
displaying the disease terrain on a computer display device; and
determining a candidate biomarker panel using the displayed protein terrain and the displayed disease terrain.


SEQUENCE LISTING (8 sequences)

SEQ ID NO: 1 - DNA, 3686 bp, Homo sapiens
atacgggagaactaaggctgaaacctcggaggaacaaccacttttgaagtgacttcgcggcgtgcgttgggtgcggactaggtggccgcggcgggagtgtgctggagcctgaagtccacgcgcgcggctgagaaccgccgggaccgcacgtgggcgccgcgcgcttcccccgcttcccaggtgggcgccggccgccaggccacctcacgtccggccccggggatgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcccgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtgaggcctgggctgggctggggctaggagggcagcagggtggcctctgggaggccaggatggcactgttggcaccgaggttgggggcagaggcccatctggcctgaactgaggctccagcatctagtggtggaccggccggtcactgcaggggtctggtggtctctgcttgcatccccaacttagctgtcccctgacccagagcctaggggatccggggcttgtacagaagagacagtccaaggggactggatcccagcagtgatgttggttgaggcagcaaacagatggcaggatgggcactgccgagaacagcattggtcccagagccctgggcatcagaccttaaccaccaggcccacagcccagcgagggagaggtcgtgaggccagctcccggggcccctgtaaccctactctcctctctccctggacctcagaggtgacacccattgggcccttccggcatgcccccagttactgtaaatgtggcccccagtgggcatggagccagtgcctgtggttgtttctccagagtcaaaagggaagtcgagggatggggcgtcgtcagctggcactgtctctgctgcagcggccacactgtactctgcactggtgtgagggcccctgcctggactgtgggaccctcctggtgctgcccaccttccctgtcctgtagccccctcggtgggcccagggcctagggcccaggatcaagtcactcatctcagaatgtccccaccaatccccgccacagcaggcgcctcgggtcccagatgtctgcagccctcagcagctgcagaccgcccctcaccaacccagagaacctgctttactttgcccagggacttcctccccatgtgaacatggggaacttcgggccctgcctggagtccttgaccgctctctgtgggccccacccactctgtcctgggaaatgaagaagcatcttccttaggtctgccctgcttgcaaatccactagcaccgaccccaccacctggttccggctctgcacgctttggggtgtggatgtcgagaggcaccacggcctcacccaggcatctgctttactctggaccataggaaacaagaccgtttggaggtttcatcaggattttgggtttttcacatttcacgctaaggagtagtggccctgacttccggtcggctggccagctgactccctagggccttcagacgtgtatgcaaatgagtgatggataaggatgagtcttggagttgcgggcagcctggagactcgtggacttaccgcctggaggcaggcccgggaaggctgctgtttactcatcgggcagccacgtgctctctggaggaagtgatagtttctgaaaccgctcagatgttttggggaaagttggagaagccgtggccttgcgagaggtggttacaccagaacctggacattggccagaagaagcttaagtgggcagacactgtttgcccagtgtttgtgcaaggatggagtgggtgtctctgcatcacccacagccgcagctgtaaggcacgctggaaggcacacgcctgccaggcagggcagtctggcgcccatgatgggagggattgacatgtttcaacaaaataatgcacttccttacctagtggcccttcacacaacttttgaatctctaaaaatccataaaatccttaaagaactgtaa

SEQ ID NO: 2 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 3 - DNA, 2780 bp, Homo sapiens
gctgcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacggtgtcccccgccgagaagtggagcgtgcacatcgccatgcaccctcaggtcaacatctacagcgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagaatgccagctgctactttgacatcgagtggcgtgaccggcgcatcacactgagggcgtccaatggcaagtttgtgacctccaagaagaatgggcagctggccgcctcggtggagacagcaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaagactccacaggcaaatactggacggtgggcagtgactccgcggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctgagccttatttctctggaagcggctaagggacggttgggggctgggagccctgggcgtgtagtgtaactggaatcttttgcctctcccagccacctcctcccagccccccaggagagctgggcacatgtcccaagcctgtcagtggccctccctggtgcactgtccccgaaacccctgcttgggaagggaagctgtcgggtgggctaggactgacccttgtggtgtttttttgggtggtggctggaaacagcccctctcccacgtggcagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggcccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcagccccaaaa

SEQ ID NO: 4 - protein, 493 aa, Homo sapiens
MTANGTAEAVQIQFGLINCGNKYLTAEAFGFKVNASASSLKKKQIWTLEQPPDEAGSAAVCLRSHLGRYLAADKDGNVTCEREVPGPDCRFLIVAHDDGRWSLQSEAHRRYFGGTEDRLSCFAQTVSPAEKWSVHIAMHPQVNIYSVTRKRYAHLSARPADEIAVDRDVPWGVDSLITLAFQDQRYSVQTADHRFLRHDGRLVARPEPATGYTLEFRSGKVAFRDCEGRYLAPSGPSGTLKAGKATKVGKDELFALEQSCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 5 - DNA, 3569 bp, Homo sapiens
accatcgtcttgggcccggggagggagagccaccttcaggcccctcgagcctcgaaccggaacctccaaatccgagacgctctgcttatgaggacctcgaaatatgccggccagtgaaaaaatcttgtggctttgagggcttttggttggccaggggcagtaaaaatctcggagagctgacaccaagtcctcccctgccacgtagcagtggtaaagtccgaagctcaaattccgagaattgagctctgttgattcttagaactggggttcttagaagtggtgatgcaagaagtttctaggaaaggccggacaccaggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaactggaa

SEQ ID NO: 6 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 7 - DNA, 2709 bp, Homo sapiens
ccctttactcctggctgcggggcgagccgggcgtctgctgcagcggccgcggtggctgaggaggcccgagaggagtcggtggcagcggcggcggcgggaccggcagcagcagcagcagcagcagcagcagcaaccactagcctcctgccccgcggcgctgccgcacgagccccacgagccgctcaccccgccgttctcagcgctgcccgaccccgctggcgcgccctcccgccgccagtcccggcagcgccctcagttgtcctccgactcgccctcggccttccgcgccagccgcagccacagccgcaacgccacccgcagccacagccacagccacagccccaggcatagccttcggcacagccccggctccggctcctgcggcagctcctctgggcaccgtccctgcgccgacatcctggaggttgggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggtcagaaccctgccatggaactgtttccttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacacaaaaaatgcacaaacaatgcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctgctggttttatctgagtgaaatactgtacaggggaataaaagagatcttatttttttttttatacttggcgttttttgaataaaaaccttttgtcttaa

SEQ ID NO: 8 - protein, 404 aa, Homo sapiens
MPHEPHEPLTPPFSALPDPAGAPSRRQSRQRPQLSSDSPSAFRASRSHSRNATRSHSHSHSPRHSLRHSPGSGSCGSSSGHRPCADILEVGMLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK